SEQUENCE LISTING (9 sequences)

SEQ ID NO: 1 - protein, 8 aa, synthetic construct
  REGION 1..8: note = Synthetic peptide
GFVFSSYD

SEQ ID NO: 2 - protein, 8 aa, synthetic construct
  REGION 1..8: note = Synthetic peptide
ISSGGGIT

SEQ ID NO: 3 - protein, 13 aa, synthetic construct
  REGION 1..13: note = Synthetic peptide
AAHYFGSSGPFAY

SEQ ID NO: 4 - protein, 6 aa, synthetic construct
  REGION 1..6: note = Synthetic peptide
ENIFSY

SEQ ID NO: 5 - protein, 9 aa, synthetic construct
  REGION 1..9: note = Synthetic peptide
QHHYGTPFT

SEQ ID NO: 6 - protein, 120 aa, synthetic construct [sequence omitted]

SEQ ID NO: 7 - protein, 107 aa, synthetic construct [sequence omitted]

SEQ ID NO: 8 - protein, 449 aa, synthetic construct
  REGION 1..449: note = Synthetic polypeptide
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 214 aa, synthetic construct
  REGION 1..214: note = Synthetic polypeptide
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0144889 A1
(43) Pub. Date: May 28, 2026

(54) CEACAM5 ANTIBODY-DRUG CONJUGATE FORMULATION

(71) Applicant: SANOFI, Paris (FR)

(72) Inventors: Jean-René AUTHELIN, Paris (FR); Fethi BENSAID, Paris (FR); Audrey BONESTEBE, Paris (FR); Sylvain HUILLE, Paris (FR); Marie LEMAN, Paris (FR); Lucie MANACHE-ALBERICI, Paris (FR)

(21) Appl. No.: 19/461,473

(22) Filed: Jan. 27, 2026

Related U.S. Application Data
(63) Continuation of application No. 17/522,443, filed on Nov. 9, 2021, now Pat. No. 12,564,642.

(30) Foreign Application Priority Data
Nov. 10, 2020 (EP) 20315449.7

(51) Int. Cl. A61K 47/68; A61K 39/00; A61P 35/00; C07K 16/28; C07K 16/30

(52) U.S. Cl. CPC A61K 47/6849; A61K 47/68033; A61K 47/6853; A61P 35/00; C07K 16/2818; C07K 16/2827; C07K 16/3023; A61K 2039/505; A61K 2039/545

(57) ABSTRACT

Provided are stabilized formulations of the human carcinoembryonic antigen-related cell adhesion molecule 5 (hCEACAM5)-targeted antibody-drug conjugate huMAb2-3-SPDB-DM4, as well as methods for making and using same.30

### CROSS-REFERENCE TO RELATED APPLICATIONS

This application is a Continuation of U.S. application Ser. No. 17/522,443 filed Nov. 9, 2021, which claims the benefit of European application number 20315449.7 filed Nov. 10, 2020, the entire disclosures of which are herein incorporated by reference.

### SEQUENCE LISTING

The instant application contains a Sequence Listing which has been submitted electronically in XML format and is hereby incorporated by reference in its entirety. Said XML file, created on Jan. 27, 2026, is named 0479_0036_US_C1_1_26_2026.xml and is 13069 bytes in size.

### FIELD

The present disclosure relates to the field of therapeutic treatment of cancers, such as non-squamous non-small cell lung cancer, which express CEACAM5. Certain aspects of the invention relate to the formulation and use of CEACAM5 antagonists, such as anti-CEACAM5 immunoconjugates, to treat cancer.

### BACKGROUND

Carcinoembryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. CEA is part of a family that belongs to the immunoglobulin superfamily. The CEA family, which consists of 18 genes, is sub-divided into two subgroups of proteins: the carcinoembryonic antigen-related cell adhesion molecule (CEACAM) subgroup and the pregnancy-specific glycoprotein subgroup (Kammerer & Zimmermann, BMC Biology 2010, 8:12).

Numerous studies have shown that CEACAM5, one of the CEACAM subgroup and identical to the originally identified CEA, is highly expressed on the surface of colorectal, gastric, lung, breast, prostate, ovary, cervix, and bladder tumor cells and weakly expressed in few normal epithelial tissues such as columnar epithelial and goblet cells in colon, mucous neck cells in the stomach, and squamous epithelial cells in esophagus and cervix (Hammarstrom et al., 2002, in “Tumor Markers, Physiology, Pathobiology, Technology and Clinical Applications” Eds. Diamandis E. P. et al., AACC Press, Washington pp 375 ff). Thus, CEACAM5 constitutes a therapeutic target suitable for tumor-specific targeting approaches, such as immunoconjugates.

huMAb2-3-SPDB-DM4 is an immunoconjugate (antibody-drug conjugate, ADC) comprising a humanized anti-CEACAM5 antibody linked to maytansinoid derivative 4 (DM4), a potent antimitotic agent that inhibits microtubule assembly. DM4 is covalently bound to the antibody through an optimized linker SPDB [N-succinimidyl-4-(2-pyridyldithio)butanoic acid] that is stable in plasma and cleavable inside cells. After binding and internalization in targeted cancer cells, huMAb2-3-SPDB-DM4 is degraded, releasing cytotoxic DM4 metabolites.

Currently huMAb2-3-SPDB-DM4 is in a number of clinical trials and is expected to be useful in the treatment of any of various types of CEACAM5-expressing cancers. For example, a recent clinical study has shown that huMAb2-3-SPDB-DM4 is effective in treating non-squamous, non-small cell lung cancer (NSQ NSCLC), a subtype that represents approximately 60% of lung cancers (see WO 2020/161214). However, ADCs such as huMAb2-3-SPDB-DM4 are difficult to formulate for long-term stability and shelf life. Thus, new formulations of huMAb2-3-SPDB-DM4 are of considerable interest.

### SUMMARY

This disclosure provides, inter alia, improved formulations of huMAb2-3-SPDB-DM4, an antibody-drug conjugate (ADC) comprised of a monoclonal antibody that binds specifically to human carcinoembryonic antigen-related cell adhesion molecule 5 (hCEACAM5), linked to N2′-deacetyl-N-2′(4-methyl-4-mercapto-1-oxopentyl)-maytansine (DM4). The formulations disclosed herein differ from, and are optimized relative to, formulations of huMAb2-3-SPDB-DM4 used in clinical trials to date.

An aspect of the instant disclosure is a pharmaceutical formulation comprising
i) anti-human carcinoembryonic antigen-related cell adhesion molecule 5 (hCEACAM5) antibody-drug conjugate (ADC),
ii) a buffering agent,
iii) a tonicity agent, and
iv) a surfactant,
   at pH about 5.0 to about 6.5,
      wherein the ADC consists of huMAb2-3-SPDB-DM4.

In certain embodiments, the pharmaceutical formulation is a lyophilized formulation.

In certain embodiments, the pharmaceutical formulation is a liquid formulation.

An aspect of the instant disclosure is a pharmaceutical formulation comprising
i) anti-human carcinoembryonic antigen-related cell adhesion molecule 5 (hCEACAM5) antibody-drug conjugate (ADC),
ii) a buffering agent,
iii) a tonicity agent,
iv) a surfactant, and
v) a chelating agent,
   at pH about 5.0 to about 6.5,
      wherein the ADC consists of huMAb2-3-SPDB-DM4.

In certain embodiments, the pharmaceutical formulation is a lyophilized formulation.

In certain embodiments, the pharmaceutical formulation is a liquid formulation.

In certain embodiments, the buffering agent is selected from the group consisting of acetate, arginine, histidine, citrate, and any combination thereof.

In certain embodiments, the tonicity agent is a polyol. In certain embodiments, the polyol is selected from the group consisting of erythritol, glycerol, lactitol, maltitol, mannitol, sorbitol, sucrose, threitol, xylitol, and any combination thereof.

In certain embodiments, the surfactant is selected from the group consisting of polysorbate 20, polysorbate 80, poloxamer 188, and any combination thereof.

In certain embodiments, the surfactant is polysorbate 80.

In certain embodiments, the chelating agent is selected from the group consisting of diethylenetriaminepentaacetic acid (DTPA), ethylene glycol-bis(β-aminoethyl ether)-N,N,N′,N′-tetraacetic acid (EGTA), ethylenediaminetetraacetic acid (EDTA), hydroxyethylenediamine triacetic acid (HEDTA), nitrilotriacetic acid, and any combination thereof. In certain embodiments, the chelating agent is EDTA.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent,
iii) a tonicity agent,
iv) a polysorbate, and
v) a chelating agent,
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, arginine, histidine, citrate, and any combination thereof,
iii) a tonicity agent,
iv) polysorbate 80, and
v) ethylenediaminetetraacetic acid (EDTA),
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof, iv) polysorbate 80, and
v) EDTA,
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a polyol,
iv) a surfactant selected from the group consisting of polysorbate 80, polysorbate 20, poloxamer 188, and any combination thereof, and
v) EDTA,
   at pH about 4.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a polyol,
iv) a surfactant selected from the group consisting of polysorbate 80, polysorbate 20, poloxamer 188, and any combination thereof, and
v) EDTA,
   at pH about 4.5 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a polyol,
iv) a surfactant selected from the group consisting of polysorbate 80, polysorbate 20, poloxamer 188, and any combination thereof, and
v) EDTA,
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof, iv) polysorbate 80, and
v) EDTA,
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) polysorbate 80, and
v) EDTA,
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) polysorbate 80, and
v) EDTA,
   at pH about 5.0 to about 6.5,
      wherein the ADC consists of huMAb2-3-SPDB-DM4.

An aspect of the instant disclosure is a pharmaceutical formulation comprising
i) anti-human carcinoembryonic antigen-related cell adhesion molecule 5 (hCEACAM5) antibody-drug conjugate (ADC),
ii) sodium acetate,
iii) sorbitol,
iv) polysorbate 80, and
v) disodium EDTA,
   at pH 5.5,
      wherein the ADC consists of huMAb2-3-SPDB-DM4.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 1-50 μM disodium EDTA,
   at pH 5.5,
      wherein the ADC consists of huMAb2-3-SPDB-DM4.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 1 μM disodium EDTA,
   at pH 5.5,
      wherein the ADC consists of huMAb2-3-SPDB-DM4.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 10 μM disodium EDTA,
   at pH 5.5,
      wherein the ADC consists of huMAb2-3-SPDB-DM4.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 50 μM disodium EDTA,
   at pH 5.5,
      wherein the ADC consists of huMAb2-3-SPDB-DM4.

An aspect of the instant disclosure is a method of treating a cancer, the method comprising administering to a subject in need thereof an effective amount of any one of the foregoing pharmaceutical formulations.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 10 μM disodium EDTA,
   at pH 5.5,
      wherein the ADC consists of huMAb2-3-SPDB-DM4.

In certain embodiments, the cancer is a high carcinoembryonic antigen-related cell adhesion molecule cancer.

In certain embodiments, the cancer is selected from the group consisting of colorectal, gastric, lung, breast, prostate, ovarian, cervical, and bladder cancer.

In certain embodiments, the cancer is selected from the group consisting of lung, breast, prostate, ovarian, cervical, and bladder cancer.

In certain embodiments, the cancer is lung cancer.

In certain embodiments, the lung cancer is non-squamous non-small cell lung carcinoma.

In certain embodiments, the subject is a human.

### BRIEF DESCRIPTION OF THE FIGURES

FIG. 1 is a general representation of huMAb2-3-SPDB-DM4 (left) and structure of the chemical portion (right).

FIG. 2 is a graph depicting SEC-UPLC T0 analysis chromatograms of prototype formulations A-H. Shown are superposition of SEC-HPLC graphs for prototypes A-H at T0. See Example 3.

FIG. 3 is a graph depicting t0 analysis chromatograms of free maytansinoids for prototypes A (acetate), C (histidine), and E (citrate). Shown are free maytansinoids for prototypes A (medium grey), C (light grey) and E (heavy grey) at T0. X-axis, minutes; Y-axis, AU.

FIG. 4A is a photographic image of SDS-PAGE at t0 of prototype formulations A-H, non-reducing conditions. FIG. 4B is a photographic image of SDS-PAGE at t0 of prototype formulations A-H, reducing conditions.

FIG. 5 is a graph depicting evolution of OD 350 nm after indicated stress. Y-axis, OD350 nm. For each stress condition, prototypes appear from A to H.

FIG. 6 is a graph depicting evolution of drug-to-antibody ratio (DAR) after indicated thermal stress. X-axis, prototype formulation.

FIG. 7 is a graph depicting evolution of aggregates during indicated thermal stress. For each prototype, stress conditions appear in following order: T0, T2w40° C., T4W40° C., T1M5° C. X-axis, prototype formulation.

FIG. 8 is a graph depicting evolution of fragments during indicated thermal stress. For each prototype, stress conditions appear in following order: T0, T2w40° C., T4W40° C., T1M5° C. X-axis, prototype formulation.

FIG. 9 is a graph depicting sum of free maytansinoids after indicated thermal stress. T0 content not visible except for Prototype D. T2W40° C. visible for all prototypes. T4W timepoint analyzed only for prototypes A, B, C, G, H. X-axis, prototype formulation; Y-axis: free maytansinoid content.

FIG. 10 is a graph depicting overlain free maytansinoid profiles after 4 weeks at 40° C. for formulations A (acetate, lower tracing) and C (histidine, upper tracing).

FIG. 11 is a graph depicting evolution of acidic isoforms after indicated thermal stress. For each prototype, timepoints are presented in following order: T0, T2W40° C., T4W40° C., T4W5° C., T4W25° C. Prototypes E and F not analyzed for T4W timepoint. X-axis, prototype formulation; Y axis, acidic isoforms %.

FIG. 12 is a graph depicting imaged capillary isoelectric focusing (iCIEF) chromatograms for prototype formulation A (acetate) at t0 (upper panel) and after 4 weeks at 40° C. (lower panel). X-axis, time (minutes).

FIG. 13 is a graph depicting overlain iCIEF chromatograms after shaking stress for formulations A (acetate), C (histidine), and E (citrate).

FIG. 14 is a graph depicting DAR evolution during thermal stress (initial formulation development; Example 2). For each timepoint, prototypes are presented from A to F. Y axis, DAR UV.

FIG. 15 is a graph depicting icIEF main charge isoforms evolution during thermal stress. For each timepoint, prototypes are presented from A to F.

FIG. 16 is a graph depicting evolution of total free maytanisoids during thermal stress. For each timepoint, prototypes are presented from A to F.

FIG. 17 is a graph depicting evolution of LMW during thermal stress. For each timepoint, prototypes are presented from A to F.

FIG. 18 is a graph depicting high accuracy liquid particle counter (HIAC) results after in-use simulation, particles≥1.5 μm. For each prototype, stresses are represented in the following order: unstressed (T0), after shaking stress, P0 (after dilution in NaCl 0.9% IV bag at 0.9 g/L), P1 (24 h after dilution in NaCl 0.9% IV bag at 0.9 g/L) and P2 (24 h after dilution in NaCl 0.9% IV bag at 0.9 g/L and perfusion through an IV infusion set with in-line filter).

FIG. 19A is a graph depicting concentration variation % after in-use simulation.

FIG. 19B is a graph depicting concentration variation after in-use simulation.

FIG. 20A is a graph depicting DAR variation (%) after in-use simulation.

FIG. 20B is a graph depicting DAR variation (%) after in-use simulation.

FIG. 21 is a graph depicting evolution of HMW during in-use simulation. For both timepoints, prototypes are represented from A to F.

FIG. 22 is a graph depicting evolution of LMW during in-use simulation. For each timepoint, prototypes are represented from A to F.

FIG. 23 is a graph depicting DAR evolution in filled vials for samples with indicated concentrations of EDTA following indicated thermal stress. T0, time zero; T2W40° C., two weeks at 40° C.; TIM T2W40° C., one month at 40° C.; T3M25° C., 3 months at 25° C. (Example 3). T3M25° C. timepoint analyzed only for the w/o EDTA and the 10 μM EDTA samples.

FIG. 24 is a graph depicting monomer % variation in filled vials for samples with indicated concentrations of EDTA following indicated thermal stress. T3M25° C. timepoint analyzed only for the w/o EDTA and the 10 μM EDTA samples.

FIG. 25 is a graph depicting high molecular weight species (HMWs) % variation in filled vials for samples with indicated concentrations of EDTA following indicated thermal stress. T3M25° C. timepoint analyzed only for the w/o EDTA and the 10 μM EDTA samples.

FIG. 26 is a graph depicting sum of other fragments evolution for samples with indicated concentrations of EDTA following indicated thermal stress.

FIG. 27 is a graph depicting H2L2% variation for samples with indicated concentrations of EDTA following indicated thermal stress (non-reducing capillary gel electrophoresis).

FIG. 28 is a graph of sum of light and heavy chains % for samples with indicated concentrations of EDTA following indicated thermal stress (reducing capillary gel electrophoresis).

FIG. 29 is a graph depicting PS80 content for samples with indicated concentrations of EDTA following indicated thermal stress. T3M25° C. timepoint not analyzed for 1 μM and 50 μM EDTA samples.

FIG. 30 is a graph depicting sum of main isoforms % in filled vials for samples with indicated concentrations of EDTA following indicated thermal stress

FIG. 31 is a graph depicting sum of low pI % in filled vials for samples with indicated concentrations of EDTA following indicated thermal stress.

### DETAILED DESCRIPTION

The present invention discloses a pharmaceutical formulation of an antibody-drug conjugate (ADC) which specifically binds human and Macacafascicularis CEACAM5 proteins. This ADC, known as huMAb2-3-SPDB-DM4, and its formulation disclosed herein, are useful in the treatment of any of various types of CEACAM5-expressing cancers, including non-squamous non-small cell lung cancer. Unexpectedly, it has been found in accordance with the instant disclosure that inclusion of modest amounts of ethylenediaminetetraacetic acid (EDTA) in formulation of huMAb2-3-SPDB-DM4 significantly increased stability of the ADC and its formulation.

Carcinoembryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. CEA is part of a family that belongs to the immunoglobulin superfamily. The CEA family, which consists of 18 genes, is subdivided into two subgroups of proteins: the carcinoembryonic antigen-related cell adhesion molecule (CEACAM) subgroup and the pregnancy-specific glycoprotein subgroup (Kammerer & Zimmermann, BMC Biology 2010, 8:12).

In humans, the CEACAM subgroup consists of 7 members: CEACAM1, CEACAM3, CEACAM4, CEACAM5, CEACAM6, CEACAM7, and CEACAM8. Numerous studies have shown that CEACAM5, identical to the originally identified CEA, is highly expressed on the surface of colorectal, gastric, lung, breast, prostate, ovary, cervix, and bladder tumor cells and only weakly expressed in few normal epithelial tissues such as columnar epithelial and goblet cells in colon, mucous neck cells in the stomach, and squamous epithelial cells in esophagus and cervix (Hammarstrom et al, 2002, in “Tumor Markers, Physiology, Pathobiology, Technology and Clinical Applications” Eds. Diamandis E. P. et al., AACC Press, Washington pp 375). Thus, CEACAM5 constitutes a therapeutic target suitable for tumor-specific targeting approaches, such as immunoconjugates.

As used herein “CEACAM5” designates the “carcinoembryonic antigen-related cell adhesion molecule 5”, also known as CD66e (Cluster of Differentiation 66e) or CEA. CEACAM5 is a glycoprotein involved in cell adhesion. CEACAM5 is highly expressed in particular on the surface of colorectal, gastric, lung and uterine tumor cells.

As used herein, “high CEACAM5 cancer” refers to any of several types of cancer including colorectal, gastric, lung, breast, prostate, ovary, cervical, and bladder cancer. In certain embodiments, “high CEACAM5 cancer” refers to any of several types of cancer including colon, lung, stomach, cervical and pancreatic cancer.

In some embodiments, the lung cancer is non-squamous non-small cell lung cancer. In certain embodiments, high CEACAM5 expressers have greater than or equal to 2+ intensity in at least 50% of expressing tumor cell population. In certain embodiments, high CEACAM5 expressers have greater than 2+ intensity in at least 50% of expressing tumor cell population. High CEACAM5 expressers represent ˜20% of lung cancers.

The ADC huMAb2-3-SPDB-DM4 is an immunoconjugate combining huMAb2-3 (anti-CEACAM5) antibody and the maytansinoid derivative 4 (DM4), a potent antimitotic agent that inhibits microtubule assembly. DM4 is covalently bound to huMAb2-3 through an optimized linker SPDB [N-succinimidyl 4-(2-pyridyldithio)-butyrate] that is stable in plasma and cleavable inside cells. After binding and internalization in targeted cancer cells, huMAb2-3-SPDB-DM4 is degraded, releasing cytotoxic DM4 metabolites.

The antibody portion of huMAb2-3-SPDB-DM4 is a human IgG1 kappa antibody having a pair of heavy chains (HC) each comprising a variable heavy (VH) domain, and a pair of light chains (LC) each comprising a variable light (VL) domain. Each VH domain comprises three complementarity-determining regions (CDRs), HCDR1, HCDR2, and HCDR3. Each VL domain comprises three complementarity-determining regions (CDRs), LCDR1, LCDR2, and LCDR3. The amino acid sequences for these polypeptides are as follows:

HCDR1
SEQ ID NO: 1
GFVFSSYD
HCDR2
SEQ ID NO: 2
ISSGGGIT
HCDR3
SEQ ID NO: 3
AAHYFGSSGPFAY
LCDR1
SEQ ID NO: 4
ENIFSY
LCDR2
NTR
LCDR3
SEQ ID NO: 5
QHHYGTPFT
VH
SEQ ID NO: 6
EVQLQESGPGLVKPGGSLSLSCAASGFVFSSYDMSWVRQTPERGLEWVAY
ISSGGGITYAPSTVKGRFTVSRDNAKNTLYLQMNSLTSEDTAVYYCAAHY
FGSSGPFAYWGQGTLVTVSS
VL
SEQ ID NO: 7
DIQMTQSPASLSASVGDRVTITCRASENIFSYLAWYQQKPGKSPKLLVYN
TRTLAEGVPSRFSGSGSGTDFSLTISSLQPEDFATYYCQHHYGTPFTFGS
GTKLEIK
HC
SEQ ID NO: 8
EVQLQESGPGLVKPGGSLSLSCAASGFVFSSYDMSWVRQTPERGLEWVAY
ISSGGGITYAPSTVKGRFTVSRDNAKNTLYLQMNSLTSEDTAVYYCAAHY
FGSSGPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
LC
SEQ ID NO: 9
DIQMTQSPASLSASVGDRVTITCRASENIFSYLAWYQQKPGKSPKLLVYN
TRTLAEGVPSRFSGSGSGTDFSLTISSLQPEDFATYYCQHHYGTPFTFGS
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

As used herein “maytansinoids” denotes maytansinoids and maytansinoid analogs. Maytansinoids are drugs that inhibit microtubule formation and that are highly toxic to mammalian cells.

Examples of suitable maytansinoids include maytansinol and maytansinol analogs.

The cytotoxic conjugates of the present invention utilize the thiol-containing maytansinoid DM4, formally termed N^{2′}-deacetyl-N-^{2′}(4-methyl-4-mercapto-1-oxopentyl)-maytansine, as the cytotoxic agent. DM4 is represented by the following structural formula (I):

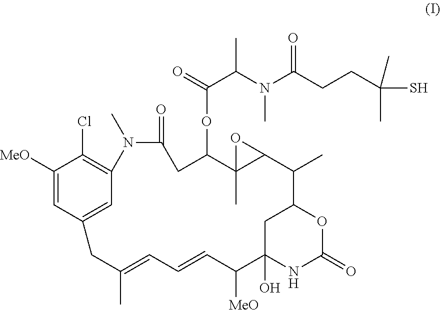 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

In some embodiments, the antibodies of the present invention are covalently attached, directly or via a cleavable or non-cleavable linker, to at least one growth inhibitory agent.

“Linker”, as used herein, means a chemical moiety comprising a covalent bond or a chain of atoms that covalently attaches a polypeptide to a drug moiety.

The conjugates may be prepared by in vitro methods. In order to link a drug or prodrug to the antibody, a linking group is used. Suitable linking groups are well known in the art and include disulfide groups, thioether groups, acid labile groups, photolabile groups, peptidase labile groups and esterase labile groups. Conjugation of an antibody of the invention with cytotoxic agents or growth inhibitory agents may be made using a variety of bifunctional protein coupling agents including but not limited to N-succinimidyl-4-(2-pyridyldithio)butanoic acid (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

The linker may be a “cleavable linker” facilitating release of the cytotoxic agent or growth inhibitory agent in the cell. For example, an acid-labile linker, a peptidase-sensitive linker, an esterase-labile linker, a photolabile linker or a disulfide-containing linker (See e.g. U.S. Pat. No. 5,208,020) may be used. The linker may be also a “non-cleavable linker” (for example SMCC linker) that might lead to better tolerance in some cases.

According to an embodiment, in the conjugate of the invention, the growth inhibitory agent is the maytansinoid DM4.

In said conjugate, the antibody is conjugated to said at least one growth inhibitory agent by a linking group. In an embodiment said linking group is a cleavable or a non-cleavable linker, such as N-succinimidyl-4-(2-pyridyldithio)butanoic acid (SPDB), 4-(Pyridin-2-yldisulfanyl)-2-sulfo-butyric acid (sulfo-SPDB), or succinimidyl (N-maleimidomethyl) cyclohexane-1-carboxylate (SMCC).

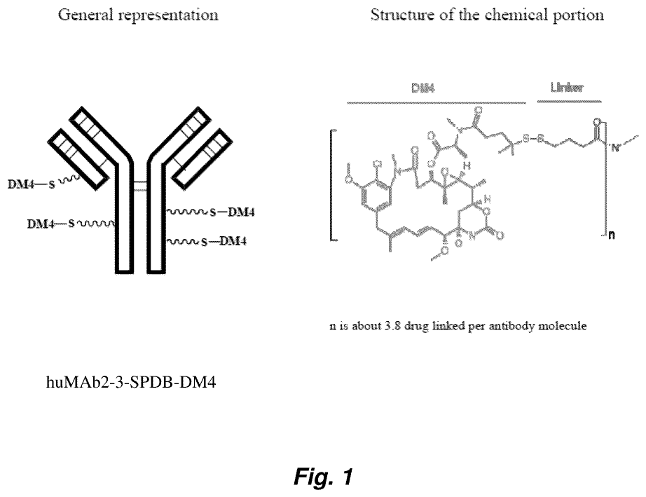

In an embodiment, the linking group is SPDB, and the conjugate consists of an antibody-SPDB-DM4 conjugate of formula (II)

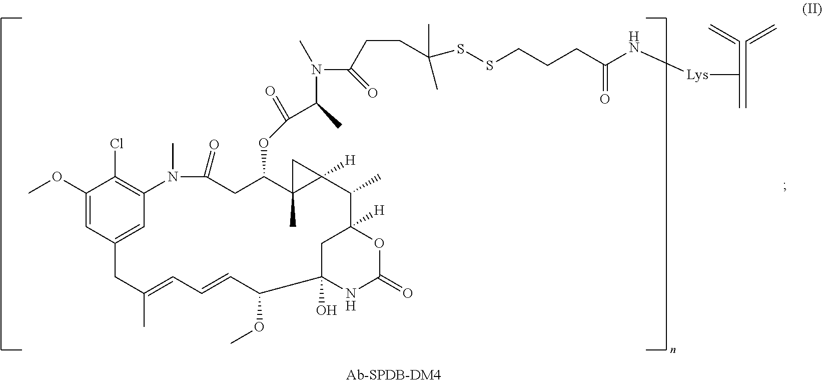

where n is an integer greater than or equal to 1. An average value of n is typically about 3.8. See also FIG. 1.

In general, the conjugate can be obtained by a process comprising the steps of:
(i) bringing into contact an optionally-buffered aqueous solution of a cell-binding agent (e.g. an antibody according to the invention) with solutions of a linker and a cytotoxic compound;
(ii) then optionally separating the conjugate which was formed in (i) from the unreacted cell-binding agent.

The aqueous solution of cell-binding agent can be buffered with buffers such as, e.g. potassium phosphate, acetate, citrate or N-2-Hydroxyethylpiperazine-N′-2-ethanesulfonic acid (HEPES buffer). The buffer depends upon the nature of the cell-binding agent. The cytotoxic compound is in solution in an organic polar solvent, e.g. dimethyl sulfoxide (DMSO) or dimethylacetamide (DMA).

The reaction temperature is usually comprised between 2° and 40° C. The reaction time can vary from 1 to 24 hours. The reaction between the cell-binding agent and the cytotoxic agent can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (ii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC). Purification by dialysis or diafiltration can also be used.

According to an embodiment, the conjugate according to the invention is characterised by a “drug-to-antibody ratio” (or “DAR”) ranging from 1 to 10, for instance from 2 to 5, in particular from 3 to 4. This is generally the case of conjugates including maytansinoid molecules.

This DAR number can vary with the nature of the antibody and of the drug (i.e. the growth-inhibitory agent) used along with the experimental conditions used for the conjugation (like the ratio growth-inhibitory agent/antibody, the reaction time, the nature of the solvent and of the cosolvent if any). Thus the contact between the antibody and the growth-inhibitory agent leads to a mixture comprising several conjugates differing from one another by different drug-to-antibody ratios; optionally the naked antibody; optionally aggregates. The DAR that is determined is thus a mean value.

A method which can be used to determine the DAR consists in measuring spectrophotometrically the ratio of the absorbance at of a solution of substantially purified conjugate at λ_{D} and 280 nm. 280 nm is a wavelength generally used for measuring protein concentration, such as antibody concentration. The wavelength λ_{D} is selected so as to allow discriminating the drug from the antibody, i.e. as readily known to the skilled person, λ_{D} is a wavelength at which the drug has a high absorbance and λ_{D} is sufficiently remote from 280 nm to avoid substantial overlap in the absorbance peaks of the drug and antibody. λ_{D} may be selected as being 252 nm in the case of maytansinoid molecules. A method of DAR calculation may be derived from Antony S. Dimitrov (ed), LLC, 2009, Therapeutic Antibodies and Protocols, vol 525, 445, Springer Science.

The absorbances for the conjugate at λ_{D} (A_{λD}) and at 280 nm (A₂₈₀) are measured either on the monomeric peak of the size exclusion chromatography (SEC) analysis (allowing to calculate the “DAR(SEC)” parameter) or using a classic spectrophotometer apparatus (allowing to calculate the “DAR(UV)” parameter). The absorbances can be expressed as follows:

$${A}_{λ D} = ( {c}_{D} \times {ε}_{D λ D} ) + ( {c}_{A} \times {ε}_{A λ D} )$$

$${A}_{280} = ( {c}_{D} \times {ε}_{D 280} ) + ( {c}_{A} \times {ε}_{A 280} )$$

wherein:
c_{D} and c_{A} are respectively the concentrations in the solution of the drug and of the antibody;
ε_{DλD} and ε_{D280} are respectively the molar extinction coefficients of the drug at λ_{D} and 280 nm; and
ε_{DλD} and ε_{A280} are respectively the molar extinction coefficients of the antibody at λ_{D} and 280 nm.

Resolution of these two equations with two unknowns leads to the following equations:

$${c}_{D} = [ ( {ε}_{A 280} \times {A}_{λ D} ) - ( {ε}_{A λ D} \times {A}_{280} ) ] / [ ( {ε}_{D λ D} \times {ε}_{A 280} ) - [ ( {ε}_{A λ D} \times {ε}_{D 280} ) ]$$

$${c}_{A} = [ {A}_{280} - ( {c}_{D} \times {ε}_{D 280} ) ] / {ε}_{A 280}$$

The average DAR is then calculated from the ratio of the drug concentration to that of the antibody:

$$DAR = {c}_{D} / {c}_{A} .$$

huMAb2-3-SPDB-DM4 is currently completing a Phase 1/Phase 2 first-in-human study for the evaluation of the safety, pharmacokinetics and antitumor activity of huMAb2-3-SPDB-DM4 in patients with advanced solid tumors (NCT02187848). A description of this study is provided in Example 1 below.

huMAb2-3-SPDB-DM4 is currently undergoing a randomized, open label Phase 3 study versus docetaxel in previously treated metastatic non-squamous non-small cell lung cancer patients with CEACAM5 positive tumors (NCT04154956).

As used herein, a “subject” refers to a mammal, including mice, rats, hamsters, Guinea pigs, rabbits, cats, dogs, sheep, goats, pigs, cows, horses, non-human primates, and humans. In certain embodiments, a subject is a human.

### Formulations

It has now been discovered, in accordance with the instant disclosure, that the formulation of huMAb2-3-SPDB-DM4 advantageously includes the ADC, acetate, sorbitol, and polysorbate 80 (PS80). It has now further been discovered, in accordance with the instant disclosure, that the formulation of huMAb2-3-SPDB-DM4 advantageously includes the ADC, acetate, sorbitol, polysorbate 80 (PS80), and EDTA. This formulation is suitable for both liquid and lyophilized forms.

It will be understood that the pharmaceutical compositions disclosed herein can be aqueous solutions containing each of the recited components in amounts and concentrations as specified.

In the case of lyophilates prepared from aqueous solutions, the recited components in amounts and concentrations as specified relate to amounts and concentrations in the formulations upon reconstitution. Liquid used for reconstitution will generally be sterile water for injection, in amounts suitable to realize the recited amounts and concentrations of the specified components.

An aspect of the instant disclosure is a pharmaceutical formulation comprising
i) anti-human carcinoembryonic antigen-related cell adhesion molecule 5 (hCEACAM5) antibody-drug conjugate (ADC),
ii) a buffering agent,
iii) a tonicity agent, and
iv) a surfactant,
   at pH about 5.0 to about 6.5,
      wherein the ADC consists of huMAb2-3-SPDB-DM4.

In certain embodiments, the pharmaceutical formulation is a lyophilized formulation.

In certain embodiments, the pharmaceutical formulation is a liquid formulation.

An aspect of the instant disclosure is a pharmaceutical formulation comprising
i) anti-human carcinoembryonic antigen-related cell adhesion molecule 5 (hCEACAM5) antibody-drug conjugate (ADC),
ii) a buffering agent,
iii) a tonicity agent,
iv) a surfactant, and
v) a chelating agent,
   at pH about 5.0 to about 6.5,
      wherein the ADC consists of huMAb2-3-SPDB-DM4.

In certain embodiments, the pharmaceutical formulation is a lyophilized formulation.

In certain embodiments, the pharmaceutical formulation is a liquid formulation.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent,
iii) a tonicity agent,
iv) a polysorbate, and
v) a chelating agent,
   at pH about 5.0 to about 6.5,
      wherein the ADC consists of huMAb2-3-SPDB-DM4.

In certain embodiments, the pharmaceutical formulation is a lyophilized formulation.

In certain embodiments, the pharmaceutical formulation is a liquid formulation.

Buffering agents that may be suitable for use in the formulations include, but are not limited to, organic acid salts such as salts of citric acid, ascorbic acid, gluconic acid, carbonic acid, tartaric acid, succinic acid, acetic acid or phthalic acid; Tris, tromethamine (tris (hydroxymethyl)-aminomethane) hydrochloride, or phosphate buffer. In addition, amino acid components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-arginine succinate, etc. These are available from a number of commercial suppliers.

Tonicity agents that may be suitable for use in the formulations include, but are not limited to, polyols including sugars (reducing and nonreducing sugars), sugar alcohols, and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and metallic salts thereof. These are available from a number of commercial suppliers.

Surfactants that may be suitable for use in the formulations include, but are not limited to, polysorbates and poloxamers. Poloxamers include, for example, poloxamer 188. These are available from a number of commercial suppliers.

Polysorbates that may be suitable for use in the formulations include, but are not limited to, polysorbate 20, polysorbate 40, polysorbate 65, polysorbate 80, polysorbate 81, and polysorbate 85. These are available from a number of commercial suppliers.

Chelating agents that may be suitable for use in the formulations include, but are not limited to, diethylenetrianiinepentaacetic acid (DTPA), ethylenediaminetetraacetic acid (EDTA), hydroxyethylenediamine triacetic acid (HEDTA), nitrilotriacetic acid, ethylene glycol-bis(β-aminoethyl ether)-N,N,N′,N′-tetraacetic acid (EGTA), and salts thereof, e.g., disodium EDTA, calcium disodium EDTA, tetrasodium EDTA. These are available from a number of commercial suppliers.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, arginine, histidine, citrate, and any combination thereof,
iii) a tonicity agent,
iv) Polysorbate 80, and
v) ethylenediaminetetraacetic acid (EDTA),
   at pH about 5.0 to about 6.5,
wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof,
iv) Polysorbate 80, and
v) EDTA,
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a polyol,
iv) a surfactant selected from the group consisting of polysorbate 80, polysorbate 20, poloxamer 188, and any combination thereof, and
v) EDTA,
   at pH about 4.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a polyol,
iv) a surfactant selected from the group consisting of polysorbate 80, polysorbate 20, poloxamer 188, and any combination thereof, and
v) EDTA,
   at pH about 4.5 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a polyol,
iv) a surfactant selected from the group consisting of polysorbate 80, polysorbate 20, poloxamer 188, and any combination thereof, and
v) EDTA,
   at pH about 5.0 to about 6.5,
      wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof,
iv) Polysorbate 80, and
v) EDTA,
   at pH about 5.0 to about 6.5,
wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) Polysorbate 80, and
v) EDTA,
   at pH about 5.0 to about 6.5,
wherein the ADC consists of huMAb2-3-SPDB-DM4.

In certain embodiments, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) Polysorbate 80, and
v) EDTA,
   at pH about 5.0 to about 6.5,
wherein the ADC consists of huMAb2-3-SPDB-DM4.

In accordance with each of the foregoing aspects and embodiments, in various certain embodiments, the pH can be about 5.0 to about 6.0. In various certain embodiments, the pH can be about 5.0 to about 5.5. In various certain embodiments, the pH can be about 5.6 to about 6.5. In various certain embodiments, the pH can be about 5.6 to about 6.0. In various certain embodiments, the pH can be about 6.1 to about 6.5.

In accordance with each of the foregoing aspects and embodiments, in various certain embodiments, the pH can be 5.0 to 6.0. In various certain embodiments, the pH can be 5.0 to 5.5. In various certain embodiments, the pH can be 5.6 to 6.5. In various certain embodiments, the pH can be 5.6 to 6.0. In various certain embodiments, the pH can be 6.1 to 6.5.

In accordance with each of the foregoing aspects and embodiments, in various certain embodiments, the pH can be about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, or about 6.5.

In accordance with each of the foregoing aspects and embodiments, in various certain embodiments, the pH can be 5.0, 5.1, 5.2, 5.3, 5.4, 5.5, 5.6, 5.7, 5.8, 5.9, 6.0, 6.1, 6.2, 6.3, 6.4, or 6.5.

In accordance with each of the foregoing aspects and embodiments, the buffering agent can be selected from acetate, histidine, citrate, and any combination thereof, at a concentration of about 5 mM to about 100 mM. In various embodiments, the buffering agent is present at a concentration of about 10 mM to about 100 mM. In various embodiments, the buffering agent is present at a concentration of about 20 mM to about 100 mM. In various embodiments, the buffering agent is present at a concentration of about 30 mM to about 100 mM. In various embodiments, the buffering agent is present at a concentration of about 40 mM to about 100 mM. In various embodiments, the buffering agent is present at a concentration of about 50 mM to about 100 mM. In various embodiments, the buffering agent is present at a concentration of about 60 mM to about 100 mM. In various embodiments, the buffering agent is present at a concentration of about 70 mM to about 100 mM. In various embodiments, the buffering agent is present at a concentration of about 80 mM to about 100 mM. In various embodiments, the buffering agent is present at a concentration of about 90 mM to about 100 mM. In various embodiments, the buffering agent is present at a concentration of about 5 mM to about 90 mM. In various embodiments, the buffering agent is present at a concentration of about 5 mM to about 80 mM. In various embodiments, the buffering agent is present at a concentration of about 5 mM to about 70 mM. In various embodiments, the buffering agent is present at a concentration of about 5 mM to about 60 mM. In various embodiments, the buffering agent is present at a concentration of about 5 mM to about 50 mM. In various embodiments, the buffering agent is present at a concentration of about 5 mM to about 40 mM. In various embodiments, the buffering agent is present at a concentration of about 5 mM to about 30 mM. In various embodiments, the buffering agent is present at a concentration of about 5 mM to about 20 mM. In various embodiments, the buffering agent is present at a concentration of about 5 mM to about 10 mM.

In accordance with each of the foregoing aspects and embodiments, the buffering agent can be selected from acetate, histidine, citrate, and any combination thereof, at a concentration of 5 mM to 100 mM. In various embodiments, the buffering agent is present at a concentration of 10 mM to 100 mM. In various embodiments, the buffering agent is present at a concentration of 20 mM to 100 mM. In various embodiments, the buffering agent is present at a concentration of 30 mM to 100 mM. In various embodiments, the buffering agent is present at a concentration of 40 mM to 100 mM. In various embodiments, the buffering agent is present at a concentration of 50 mM to 100 mM. In various embodiments, the buffering agent is present at a concentration of 60 mM to 100 mM. In various embodiments, the buffering agent is present at a concentration of 70 mM to 100 mM. In various embodiments, the buffering agent is present at a concentration of 80 mM to 100 mM. In various embodiments, the buffering agent is present at a concentration of 90 mM to 100 mM. In various embodiments, the buffering agent is present at a concentration of 5 mM to 90 mM. In various embodiments, the buffering agent is present at a concentration of 5 mM to 80 mM. In various embodiments, the buffering agent is present at a concentration of 5 mM to 70 mM. In various embodiments, the buffering agent is present at a concentration of 5 mM to 60 mM. In various embodiments, the buffering agent is present at a concentration of 5 mM to 50 mM. In various embodiments, the buffering agent is present at a concentration of 5 mM to 40 mM. In various embodiments, the buffering agent is present at a concentration of 5 mM to 30 mM. In various embodiments, the buffering agent is present at a concentration of 5 mM to 20 mM. In various embodiments, the buffering agent is present at a concentration of 5 mM to 10 mM.

In accordance with each of the foregoing aspects and embodiments, in various embodiments, the buffering agent is present at a concentration of about 5 mM. In various embodiments, the buffering agent is present at a concentration of about 10 mM. In various embodiments, the buffering agent is present at a concentration of about 15 mM. In various embodiments, the buffering agent is present at a concentration of about 20 mM. In various embodiments, the buffering agent is present at a concentration of about 25 mM. In various embodiments, the buffering agent is present at a concentration of about 30 mM. In various embodiments, the buffering agent is present at a concentration of about 40 mM. In various embodiments, the buffering agent is present at a concentration of about 50 mM. In various embodiments, the buffering agent is present at a concentration of about 60 mM. In various embodiments, the buffering agent is present at a concentration of about 70 mM. In various embodiments, the buffering agent is present at a concentration of about 80 mM. In various embodiments, the buffering agent is present at a concentration of about 90 mM. In various embodiments, the buffering agent is present at a concentration of about 100 mM.

In accordance with each of the foregoing aspects and embodiments, in various embodiments, the buffering agent is present at a concentration of 5 mM. In various embodiments, the buffering agent is present at a concentration of 10 mM. In various embodiments, the buffering agent is present at a concentration of 15 mM. In various embodiments, the buffering agent is present at a concentration of 20 mM. In various embodiments, the buffering agent is present at a concentration of 25 mM. In various embodiments, the buffering agent is present at a concentration of 30 mM. In various embodiments, the buffering agent is present at a concentration of 40 mM. In various embodiments, the buffering agent is present at a concentration of 50 mM. In various embodiments, the buffering agent is present at a concentration of 60 mM. In various embodiments, the buffering agent is present at a concentration of 70 mM. In various embodiments, the buffering agent is present at a concentration of 80 mM. In various embodiments, the buffering agent is present at a concentration of 90 mM. In various embodiments, the buffering agent is present at a concentration of 100 mM.

In accordance with each of the foregoing aspects and embodiments, in certain embodiments the tonicity agent is selected from erythritol, glycerol, lactitol, maltitol, mannitol, sorbitol, sucrose, threitol, xylitol, and any combination thereof, at a concentration of about 1% (w/v) to about 50% (w/v). In certain embodiments, the tonicity agent is present at a concentration of about 5% (w/v) to about 50% (w/v). In certain embodiments, the tonicity agent is present at a concentration of about 10% (w/v) to about 50% (w/v). In certain embodiments, the tonicity agent is present at a concentration of about 15% (w/v) to about 50% (w/v). In certain embodiments, the tonicity agent is present at a concentration of about 20% (w/v) to about 50% (w/v). In certain embodiments, the tonicity agent is present at a concentration of about 25% (w/v) to about 50% (w/v). In certain embodiments, the tonicity agent is present at a concentration of about 30% (w/v) to about 50% (w/v). In certain embodiments, the tonicity agent is present at a concentration of about 40% (w/v) to about 50% (w/v). In certain embodiments, the tonicity agent is present at a concentration of about 1% (w/v) to about 40% (w/v). In certain embodiments, the tonicity agent is present at a concentration of about 1% (w/v) to about 30% (w/v). In certain embodiments, the tonicity agent is present at a concentration of about 1% (w/v) to about 25% (w/v). In certain embodiments, the tonicity agent is present at a concentration of about 1% (w/v) to about 20% (w/v). In certain embodiments, the tonicity agent is present at a concentration of about 1% (w/v) to about 15% (w/v). In certain embodiments, the tonicity agent is present at a concentration of about 1% (w/v) to about 10% (w/v). In certain embodiments, the tonicity agent is present at a concentration of about 1% (w/v) to about 5% (w/v).

In accordance with each of the foregoing aspects and embodiments, in certain embodiments the tonicity agent is selected from erythritol, glycerol, lactitol, maltitol, mannitol, sorbitol, sucrose, threitol, xylitol, and any combination thereof, at a concentration of 1% (w/v) to 50% (w/v). In certain embodiments, the tonicity agent is present at a concentration of 5% (w/v) to 50% (w/v). In certain embodiments, the tonicity agent is present at a concentration of 10% (w/v) to 50% (w/v). In certain embodiments, the tonicity agent is present at a concentration of 15% (w/v) to 50% (w/v). In certain embodiments, the tonicity agent is present at a concentration of 20% (w/v) to 50% (w/v). In certain embodiments, the tonicity agent is present at a concentration of 25% (w/v) to 50% (w/v). In certain embodiments, the tonicity agent is present at a concentration of 30% (w/v) to 50% (w/v). In certain embodiments, the tonicity agent is present at a concentration of 40% (w/v) to 50% (w/v). In certain embodiments, the tonicity agent is present at a concentration of 1% (w/v) to 40% (w/v). In certain embodiments, the tonicity agent is present at a concentration of 1% (w/v) to 30% (w/v). In certain embodiments, the tonicity agent is present at a concentration of 1% (w/v) to 25% (w/v). In certain embodiments, the tonicity agent is present at a concentration of 1% (w/v) to 20% (w/v). In certain embodiments, the tonicity agent is present at a concentration of 1% (w/v) to 15% (w/v). In certain embodiments, the tonicity agent is present at a concentration of 1% (w/v) to 10% (w/v). In certain embodiments, the tonicity agent is present at a concentration of 1% (w/v) to 5% (w/v).

In accordance with each of the foregoing aspects and embodiments, in certain embodiments the tonicity agent is selected from erythritol, glycerol, lactitol, maltitol, mannitol, sorbitol, sucrose, threitol, xylitol, and any combination thereof, at a concentration of about 1% (w/v). In certain embodiments, the tonicity agent is present at a concentration of about 2% (w/v). In certain embodiments, the tonicity agent is present at a concentration of about 3% (w/v). In certain embodiments, the tonicity agent is present at a concentration of about 4% (w/v). In certain embodiments, the tonicity agent is present at a concentration of about 5% (w/v). In certain embodiments, the tonicity agent is present at a concentration of about 10% (w/v). In certain embodiments, the tonicity agent is present at a concentration of about 15% (w/v). In certain embodiments, the tonicity agent is present at a concentration of about 20% (w/v). In certain embodiments, the tonicity agent is present at a concentration of about 25% (w/v). In certain embodiments, the tonicity agent is present at a concentration of about 30% (w/v). In certain embodiments, the tonicity agent is present at a concentration of about 40% (w/v). In certain embodiments, the tonicity agent is present at a concentration of about 50% (w/v).

In accordance with each of the foregoing aspects and embodiments, in certain embodiments the tonicity agent is selected from erythritol, glycerol, lactitol, maltitol, mannitol, sorbitol, sucrose, threitol, xylitol, and any combination thereof, at a concentration of 1% (w/v). In certain embodiments, the tonicity agent is present at a concentration of 2% (w/v). In certain embodiments, the tonicity agent is present at a concentration of 3% (w/v). In certain embodiments, the tonicity agent is present at a concentration of 4% (w/v). In certain embodiments, the tonicity agent is present at a concentration of 5% (w/v). In certain embodiments, the tonicity agent is present at a concentration of 10% (w/v). In certain embodiments, the tonicity agent is present at a concentration of 15% (w/v). In certain embodiments, the tonicity agent is present at a concentration of 20% (w/v). In certain embodiments, the tonicity agent is present at a concentration of 25% (w/v). In certain embodiments, the tonicity agent is present at a concentration of 30% (w/v). In certain embodiments, the tonicity agent is present at a concentration of 40% (w/v). In certain embodiments, the tonicity agent is present at a concentration of 50% (w/v).

An aspect of the instant disclosure is a pharmaceutical formulation comprising
i) anti-human carcinoembryonic antigen-related cell adhesion molecule 5 (hCEACAM5) antibody-drug conjugate (ADC),
ii) Sodium Acetate,
iii) Sorbitol,
iv) Polysorbate 80, and
v) disodium EDTA,
   at pH 5.5,
wherein the ADC consists of huMAb2-3-SPDB-DM4.

In certain embodiments, the pharmaceutical formulation comprises about 1 to about 50 mg/mL huMAb2-3-SPDB-DM4. In certain embodiments, the pharmaceutical formulation comprises about 2 to about 40 mg/mL huMAb2-3-SPDB-DM4. In certain embodiments, the pharmaceutical formulation comprises about 3 to about 30 mg/mL huMAb2-3-SPDB-DM4. In certain embodiments, the pharmaceutical formulation comprises about 4 to about 20 mg/mL huMAb2-3-SPDB-DM4. In certain embodiments, the pharmaceutical formulation comprises about 5 to about 10 mg/mL huMAb2-3-SPDB-DM4. In certain embodiments, the pharmaceutical formulation comprises about 5 mg/mL huMAb2-3-SPDB-DM4.

In certain embodiments, the pharmaceutical formulation comprises 1-50 mg/mL huMAb2-3-SPDB-DM4. In certain embodiments, the pharmaceutical formulation comprises 2-40 mg/mL huMAb2-3-SPDB-DM4. In certain embodiments, the pharmaceutical formulation comprises 3-30 mg/mL huMAb2-3-SPDB-DM4. In certain embodiments, the pharmaceutical formulation comprises 4-20 mg/mL huMAb2-3-SPDB-DM4. In certain embodiments, the pharmaceutical formulation comprises 5-10 mg/mL huMAb2-3-SPDB-DM4. In certain embodiments, the pharmaceutical formulation comprises 5 mg/mL huMAb2-3-SPDB-DM4. In certain embodiments, the pharmaceutical formulation comprises 10 mg/mL huMAb2-3-SPDB-DM4.

In certain embodiments, the pharmaceutical composition comprises about 1 to about 50 μM disodium EDTA. In certain embodiments, the pharmaceutical composition comprises about 1 to about 40 μM disodium EDTA. In certain embodiments, the pharmaceutical composition comprises about 1 to about 30 μM disodium EDTA. In certain embodiments, the pharmaceutical composition comprises about 1 to about 20 μM disodium EDTA. In certain embodiments, the pharmaceutical composition comprises about 1 to about 10 μM disodium EDTA. In certain embodiments, the pharmaceutical composition comprises about 10 to about 50 μM disodium EDTA. In certain embodiments, the pharmaceutical composition comprises about 10 to about 40 μM disodium EDTA. In certain embodiments, the pharmaceutical composition comprises about 10 to about 20 μM disodium EDTA. In certain embodiments, the pharmaceutical composition comprises about 10 μM disodium EDTA. In certain embodiments, the pharmaceutical composition comprises about 20 μM disodium EDTA. In certain embodiments, the pharmaceutical composition comprises about 30 μM disodium EDTA. In certain embodiments, the pharmaceutical composition comprises about 40 μM disodium EDTA. In certain embodiments, the pharmaceutical composition comprises about 50 μM disodium EDTA.

In certain embodiments, the pharmaceutical composition comprises 1 to 50 μM disodium EDTA. In certain embodiments, the pharmaceutical composition comprises 1 to 40 μM disodium EDTA. In certain embodiments, the pharmaceutical composition comprises 1 to 30 μM disodium EDTA. In certain embodiments, the pharmaceutical composition comprises 1 to 20 μM disodium EDTA. In certain embodiments, the pharmaceutical composition comprises 1 to 10 μM disodium EDTA. In certain embodiments, the pharmaceutical composition comprises 10 to 50 μM disodium EDTA. In certain embodiments, the pharmaceutical composition comprises 10 to 40 μM disodium EDTA. In certain embodiments, the pharmaceutical composition comprises 10 to 20 μM disodium EDTA. In certain embodiments, the pharmaceutical composition comprises 10 μM disodium EDTA. In certain embodiments, the pharmaceutical composition comprises 20 μM disodium EDTA. In certain embodiments, the pharmaceutical composition comprises 30 μM disodium EDTA. In certain embodiments, the pharmaceutical composition comprises 40 μM disodium EDTA. In certain embodiments, the pharmaceutical composition comprises 50 μM disodium EDTA.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 1-50 μM disodium EDTA,
   at pH 5.5,
wherein the ADC consists of huMAb2-3-SPDB-DM4.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 1 μM disodium EDTA,
   at pH 5.5,
wherein the ADC consists of huMAb2-3-SPDB-DM4.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 10 μM disodium EDTA,
   at pH 5.5,
wherein the ADC consists of huMAb2-3-SPDB-DM4.

In certain embodiments, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 50 μM disodium EDTA,
   at pH 5.5,
wherein the ADC consists of huMAb2-3-SPDB-DM4.

The formulations disclosed herein are generally suitable for administration to a subject by injection or infusion, for example by intravenous injection or infusion. The formulations disclosed herein are generally suitable for administration to a human subject by injection or infusion, for example by intravenous injection or infusion.

### Methods

An aspect of the instant disclosure is a method of treating a cancer, the method comprising administering to a subject in need thereof an effective amount of any one of the foregoing pharmaceutical formulations.

In accordance with such method, in certain embodiments, the pharmaceutical formulation comprises
i) anti-human carcinoembryonic antigen-related cell adhesion molecule 5 (hCEACAM5) antibody-drug conjugate (ADC),
ii) a buffering agent,
iii) a tonicity agent,
iv) a polysorbate, and
v) a chelating agent,
   at pH about 5.0 to about 6.5,
wherein the ADC consists of huMAb2-3-SPDB-DM4.

In certain embodiments in accordance with the method, the pharmaceutical formulation comprises
i) anti-human carcinoembryonic antigen-related cell adhesion molecule 5 (hCEACAM5) antibody-drug conjugate (ADC),
ii) a buffering agent selected from acetate, arginine, histidine, citrate, and any combination thereof,
iii) a tonicity agent,
iv) Polysorbate 80, and
v) ethylenediaminetetraacetic acid (EDTA),
   at pH about 5.0 to about 6.5,
wherein the ADC consists of huMAb2-3-SPDB-DM4.

In certain embodiments in accordance with the method, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof,
iv) Polysorbate 80, and
v) EDTA,
   at pH about 5.0 to about 6.5,
wherein the ADC consists of huMAb2-3-SPDB-DM4.

In certain embodiments in accordance with the method, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof,
iv) Polysorbate 80, and
v) EDTA,
   at pH about 5.0 to about 6.5,
wherein the ADC consists of huMAb2-3-SPDB-DM4.

In certain embodiments in accordance with the method, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) Polysorbate 80, and
v) EDTA,
   at pH about 5.0 to about 6.5,
wherein the ADC consists of huMAb2-3-SPDB-DM4.

In certain embodiments in accordance with the method, the pharmaceutical formulation comprises
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) Polysorbate 80, and
v) EDTA,
   at pH about 5.0 to about 6.5,
wherein the ADC consists of huMAb2-3-SPDB-DM4.

In certain embodiments in accordance with the method, the pharmaceutical formulation comprises
i) anti-human carcinoembryonic antigen-related cell adhesion molecule 5 (hCEACAM5) antibody-drug conjugate (ADC),
ii) Sodium Acetate,
iii) Sorbitol,
iv) Polysorbate 80, and
v) disodium EDTA,
   at pH 5.5,
wherein the ADC consists of huMAb2-3-SPDB-DM4.

In certain embodiments in accordance with the method, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 1-50 μM disodium EDTA,
   at pH 5.5,
wherein the ADC consists of huMAb2-3-SPDB-DM4.

In certain embodiments in accordance with the method, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 1 μM disodium EDTA,
   at pH 5.5,
wherein the ADC consists of huMAb2-3-SPDB-DM4.

In certain embodiments in accordance with the method, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 10 μM disodium EDTA,
   at pH 5.5,
wherein the ADC consists of huMAb2-3-SPDB-DM4.

In certain embodiments in accordance with the method, the pharmaceutical formulation comprises
i) 5 mg/mL anti-human carcinoembryonic antigen-related cell adhesion molecule 5 (hCEACAM5) antibody-drug conjugate (ADC),
ii) Sodium Acetate 10 mM,
iii) Sorbitol 5% (w/v),
iv) 0.04% (w/v) Polysorbate 80, and
v) 50 μM disodium EDTA,
   at pH 5.5,
wherein the ADC consists of huMAb2-3-SPDB-DM4.

In certain embodiments, the cancer is a high carcinoembryonic antigen-related cell adhesion molecule cancer.

In certain embodiments, the cancer is selected from the group consisting of colorectal, gastric, lung, breast, prostate, ovary, cervical, and bladder cancer.

In certain embodiments, the cancer is selected from the group consisting of lung, breast, prostate, ovary, cervical, and bladder cancer.

In certain embodiments, the cancer is lung cancer.

In certain embodiments, the lung cancer is non-squamous non-small cell lung carcinoma.

In certain embodiments, the subject is a human.

In certain embodiments, the cancer is selected from the group consisting of colorectal, gastric, lung, breast, prostate, ovary, cervical, and bladder cancer.

In certain embodiments, the cancer is selected from the group consisting of lung, breast, prostate, ovary, cervical, and bladder cancer.

In certain embodiments, the cancer is lung cancer.

In certain embodiments, the lung cancer is non-squamous non-small cell lung carcinoma.

Non-small cell lung cancer (NSCLC) is the most common type of lung cancer. It usually grows and spreads more slowly than less-common small cell lung cancer. There are three main subtypes of non-small cell lung cancer, including squamous cell carcinoma (25% of lung cancers), adenocarcinoma (40% of lung cancers), and large cell carcinoma (10% of lung cancers). Non-squamous non-small cell lung cancer thus includes adenocarcinoma (40% of lung cancers) and large cell carcinoma (10% of lung cancers).

In certain embodiments, the subject is a human.

### EXAMPLES

### Example 1. Initial Formulation of huMAb2-3-SPDB-DM4

huMAb2-3-SPDB-DM4 (see FIG. 1) was initially developed as a concentrate for solution for infusion. The dosage form was a solution at 5 mg/mL for single use in glass vial stored at 5° C. The drug substance was developed as a bulk frozen solution at −20° C. in polycarbonate bottle, representative of other containers. The aim of this initial study was to identify a set of pH buffering systems and stabilizing excipients. A total of 8 formulations with the combined excipients were tested in thermal stress at 40° C., 25° C. and 5° C. and upon freeze/thaw stress and mechanical stress (shaking).

Prototypes are presented in Table 1.

TABLE 1
Combination of excipients around pH 6.0 for
huMAb2-3-SPDB-DM4 evaluation

| N° | Buffer System | Excipient | pH |
|---|---|---|---|
| A | Acetate 10 mM | Sucrose 2.5% | 5.5 |
| | | Mannitol 3.75% | |
| | | PS80 0.005% | |
| B | Acetate 10 mM | Sucrose 2.5% | 5.5 |
| | | Mannitol 3.75% | |
| | | PS80 0.1% | |
| C | Histidine 10 | Sucrose 5.0% | 5.5 |
| | mM | Glycine 130 mM | |
| | | PS80 0.1% | |
| D | Histidine 10 | Sucrose 10% | 6.5 |
| | mM | PS80 0.1% | |
| E | Citrate 10 mM | Sucrose 10% | 6.0 |
| | | PS80 0.1% | |
| F | Acetate 10 mM | Sucrose 10% | 6.0 |
| | | PS80 0.1% | |
| G | Acetate 10 mM | Sucrose 10% | 5.5 |
| | | PS80 0.1% | |
| H | Acetate 10 mM | Sucrose 10% | 5.5 |
| | | PS20 0.1% | |

### UF/DF Procedure

The starting material was provided in histidine buffer. To manufacture acetate-based prototypes (prototypes A, B, F, G, H), the starting material was processed for diafiltration on Cogent μScale system with Pellicon 3® cassette against Acetate 10 mM pH 5.5 (3 membranes of 88 cm², 68.7 g of protein/in² membrane). To manufacture citrate-based prototype (prototype E), the starting material was processed for diafiltration on Cogent μScale system with Pellicon 3® cassette against Citrate 10 mM pH 6.0 (1 membrane of 88 cm², 51.4 g of protein/m² membrane).

In both cases, the diafiltration was stopped after passing 10 volumes of diafiltration buffer. After the diafiltration, concentration was targeted to 5 mg/mL by adding appropriate concentrated 2× solution as described in Table 3.

TABLE 3
Concentrated excipient solution 2× recipes

| N° | Buffer System | Excipient | Targeted pH |
|---|---|---|---|
| A | Acetate 10 mM | Sucrose 5% | 5.5 |
| | | Mannitol 7.5% | |
| | | PS80 0.01% | |
| B | Acetate 10 mM | Sucrose 5% | 5.5 |
| | | Mannitol 7.5% | |
| | | PS80 0.2% | |
| C | Histidine 10 mM | Sucrose 10% | 5.5 |
| | | Glycine 260 mM | |
| | | PS80 0.2% | |
| D | Histidine 10 mM | Sucrose 20% | 6.5 |
| | | PS80 0.2% | |
| E | Citrate 10 mM | Sucrose 20% | 6.0 |
| | | PS80 0.2% | |
| F | Acetate 10 mM | Sucrose 20% | 6.0 |
| | | PS80 0.2% | |
| G | Acetate 10 mM | Sucrose 20% | 5.5 |
| | | PS80 0.2% | |
| H | Acetate 10 mM | Sucrose 20% | 5.5 |
| | | PS20 0.2% | |

### Filtration 0.2 μm

All formulations were filtered on 0.2 μm filters after formulation with concentrated 2× solutions and collected on Nalgene® (HDPE) bottles before filling.

### Stress Conditions

### Thermal Stress

Formulation assays A to H were stored at 40° C. for 2 weeks; at 5° C., 25° C. and 40° C. for 4 weeks before analyses.

### Freeze/Thaw Cycles

Formulation assays A to H were frozen at −20° C. or −80° C. and thawed at room temperature. 3 cycles of freeze/thaw procedure were performed and the formulations were then analyzed.

### Shaking Stress

Formulation assays A to H were shaken in vials during 15 hours via orbital shaking at a speed of 350 rpm.

### Analytical Methods

The following analytical methods were performed during the study:

Visual inspection for appearance (clarity, color and particles): the vials are inspected 5 s on white surface then on black surface of the visual inspection table.

Protein Concentration and DAR by UV measurement: OD at 280 nm and 254 nm measured using below absorptivity. Dilution factor of the solution for examination: F=10.
Absorbance of the solution for examination at 254 nm: A254D
Absorbance of the solution for examination at 280 nm: A280D
Molecular weight of naked antibody h4D4=144522 g/mol
Molecular weight of DM4=780 g/mol
Molar extinction coefficient of DM4 at 280 nm=4927 L*mol⁻¹*cm⁻¹
Molar extinction coefficient of naked antibody h4D4 at 280 nm=201400 L*mol⁻¹*cm⁻¹
A254/A280 of h4D4=0.43
A254/A280 of DM4=4.83
[Drug] (M)=[A254−(0.43×A280)]/[(4.83×4927)−(0.43×4927)]
[Drug] (mg/mL)=[Drug] (M)×780×F
[Protein] (M)=[A280−4927×[Drug] (M)]/201400
[Protein] (mg/mL)=[Protein] (M)×144522×F

Turbidity (OD at 350 nm) for opalescence characterization: the absorbance of the solution is measured at 350 nm, on 0.8 mL of non-diluted solution.

Protein purity by SEC-UPLC: Size-exclusion ultra-performance liquid chromatography (SEC-UPLC) was performed for samples A, B, G and H, at T0, after 2 and 4 weeks at 40° C. and after 4 weeks at 5° C.

### SEC-UPLC Conditions:

Column: BEH 200, 1.7 μm, 300 mm*4.6 mm
Flow: 0.30 mL/min
Detection: 230 nm
Column temperature: 30° C.±2° C.
Syringe temperature: 5° C.±0.3° C.
Volume of injection: 4 μL
Analysis time: 17 min

Phase mobile D-PBS (2×): prepared with D-PBS (10×) (Dulbecco's Phosphate Buffered Saline (D-PBS) (10×)), reference Invitrogen: 14200083: dilute this solution in ⅕ with water for injection (WFI) and filtrate on 0.22 μm. A DS batch was used as reference within each sequences (injection of 5 μL at 5 g/L).

### Protein Purity by SDS-PAGE:

Measured only at T0 and after 2 at weeks 40° C. Samples are prepared in reduced and non-reduced conditions (by addition of nem) and migrated on a 4-12% Bis-Tris gel. The migration buffer is manufactured by diluting 50 mL of Nu-Page MOPS SDS buffer 20× in sqf 1000 mL of purified water. Migration parameters are 135V during 90 minutes. Gels are colored with a Bleu rotiphorese solution.

Charge Heterogeneity by iCIEF:

4 weeks analysis realized only for acetate formulations at pH 5.5.

IcIEF is performed using a cIEF Cartbridge FC-coated with a Convergent Biosciences iCE280 imaged capillary electrophoresis system. Samples are diluted in a Master mix containing Pharmalyte 3-10, Pharmalyte 8-10.5, 4M Urea, 0.35% Methyl Cellulose 1% and pI markers.

Each sample is injected into the capillary with an autosampler. After focusing, the detection is performed by a CCD camera which realizes a direct measurement of the 280 nm UV absorbance. The iCE280 software calculates the apparent pI of each separated peak and Empower allows a quantification of the isoform by normalization per area.

### Free Maytansinoids:

4 weeks analysis realized only for formulations at pH 5.5.

Free maytansinoids are measured by RP-HPLC with two columns: a first column to separate the protein from the free maytansinoids, and a second column to separate free maytansinoids species and detect them on a PDA detector. Mobile phase A is a 0.1% trifluoroacetic acid solution in purified water. Mobile phase B is a 0.08% trifluoroacetic acid solution in acetonitrile. DM4 solutions are used as reference.

DSC (differential scanning calorimetry) for thermal stability performed only at T0.

DLS (dynamic light scattering) for colloidal aggregation state: Size average of particles and distribution. 4 weeks analysis realized only for formulations at pH 5.5.

FCM (flow cytometry) for the sub-visible aggregation state: Morphological analysis and counting of particles. 4 weeks analysis realized only for formulations at pH 5.5.

### Results and Discussion

Observation and SEC Analysis of DS in Histidine Buffer and after TFF in Acetate and Citrate Buffers

At reception the batch of DS in histidine buffer post filtration 0.2 μm was slightly opalescent with particles. The results after UFDF in acetate and citrate buffers are given in Table 4.

TABLE 4
UFDF results

| | Yield | | | | | |
|---|---|---|---|---|---|---|
| | of | Process | Concentration | Purity | | |
| | TFF | time and | Post UFDF | (%) | HMW | LMW |
| Batch | (%) | volume | (mg/mL) | Monomer | (%) | (%) |
| After TFF | 92.0 | 46 min | 11.3 | 94.2 | 3.0 | 2.75 |
| in Acetate | | for 160 mL | | | | |
| Buffer | | | | | | |
| pH 5.5 | | | | | | |
| After TFF | 81.5 | 68 min | 12.7 | 94.4 | 3.1 | 2.5 |
| in Citrate | | for 40 mL | | | | |
| Buffer | | | | | | |
| pH 6.0 | | | | | | |

Acetate buffer seemed better with regards to opalescence and yield.

### Stress Studies

### Analysis at T0

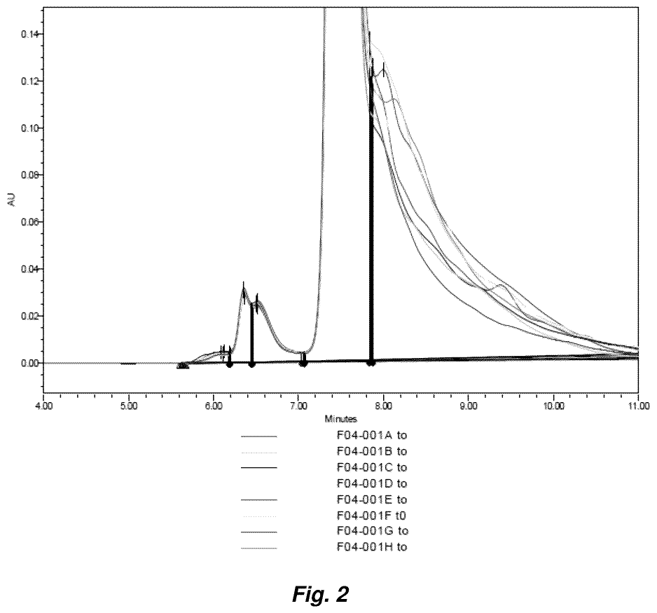

Prototypes are equivalent at T0 for visual inspection, FCM, DLS, SEC-UPLC (FIG. 2) and icIEF analytical assays:
regarding visual inspection, all the formulations were very slightly opalescent and contained no visible particles.
regarding DLS, all the formulations were polydisperse with a PDI superior to 30%.

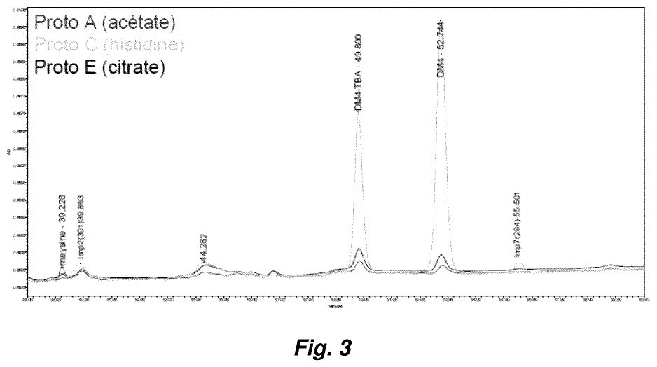

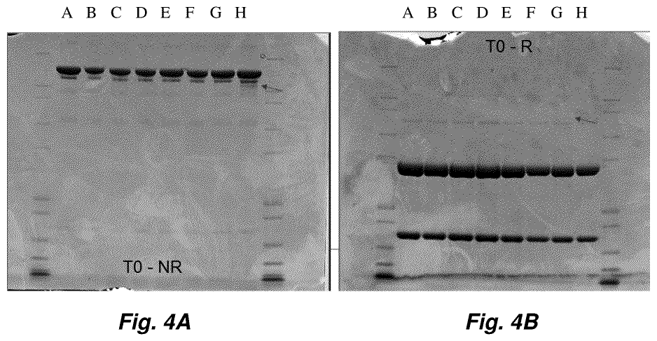

Some differences between prototypes could be observed at T0:
Regarding results of DSC, Tm was higher with Acetate buffer (pH 5.5 or 6).
For free maytansinoids, total content was lower in acetate or citrate buffers and higher in histidine buffer (<0.1% for acetate and citrate buffers and around 0.6% for histidine buffer). See FIG. 3.
Concerning results of SDS PAGE, formulations were similar except formulations G and H which had a different profile. Formulation H in non-reduced condition presents a supplementary band at 127 kDa, and Formulation G in reduced condition presents a supplementary band at 89 kDa (FIGS. 4A and 4B).

### Thermal Stress

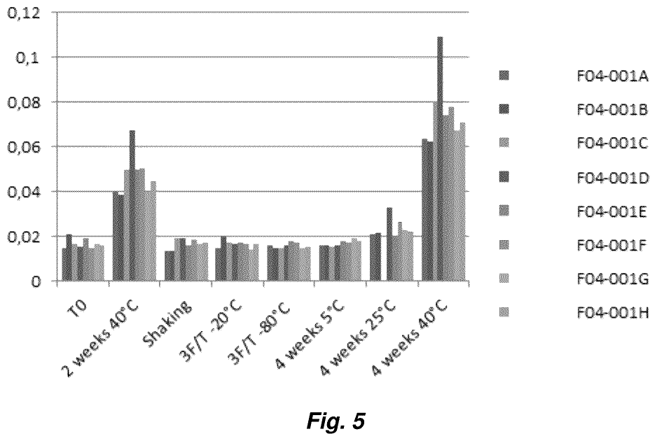

After 4 weeks at 5° C. and 25° C., all prototypes were similar for analyzed parameters (visual inspection, SEC-HPLC, FCM, DLS, free maytansinoids, ic IEF) except prototype C after 4W25° C. that is suspected of contamination (as related to pH, concentration, DLS, FCM). See turbidity results FIG. 5.

After 2 weeks and 4 weeks at 40° C., prototypes are equivalent for protein concentration, SDS-PAGE, visual inspection analytical assays:
After 2 weeks at 40° C., a new band at 145 kDa was detected for all formulations in SDS PAGE reduced conditions.
After 2 weeks at 40° C., with regard to visual inspection, all the formulations were very slightly opalescent. Nevertheless, after 40° C. stress, no real evolution was observed versus T0.

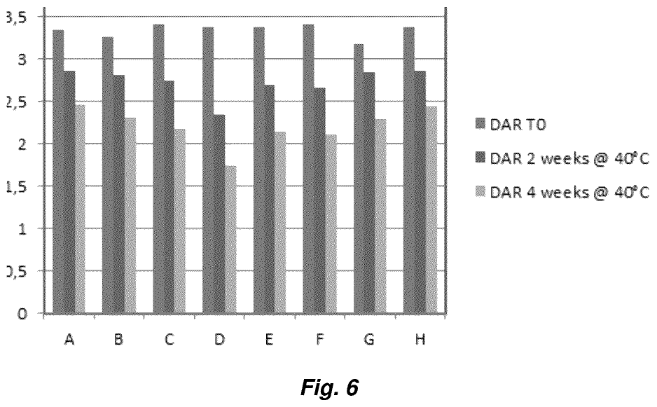

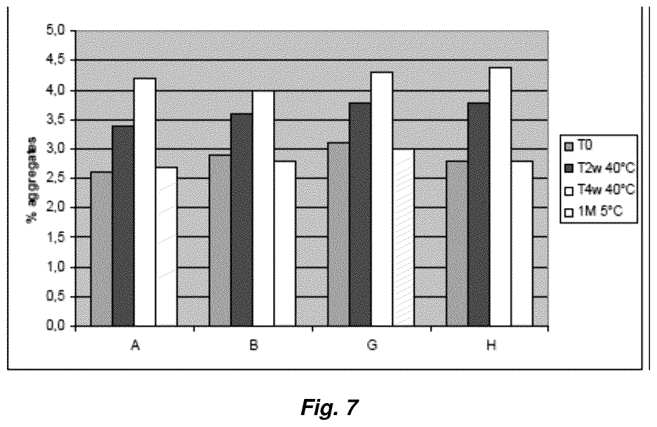

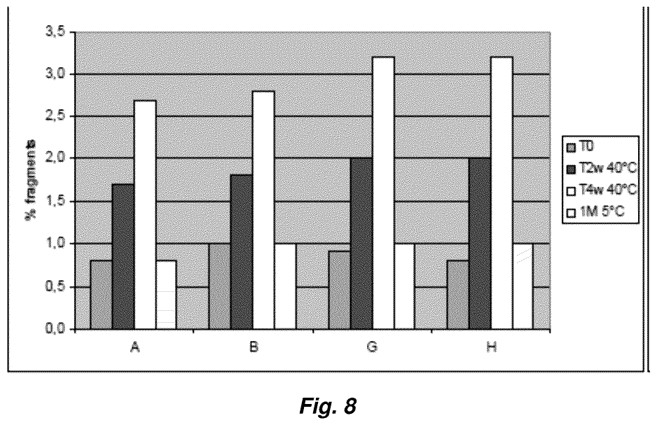

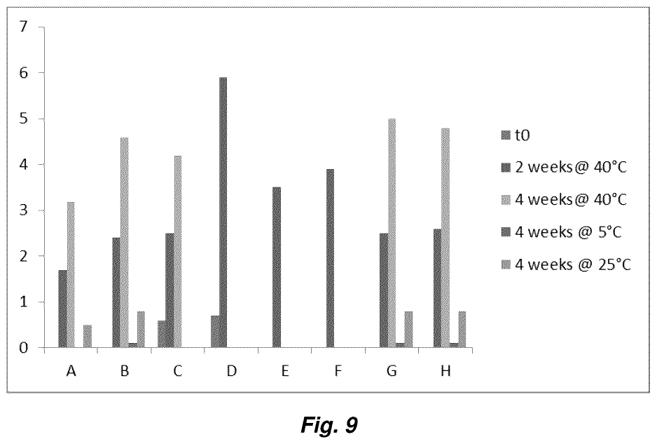

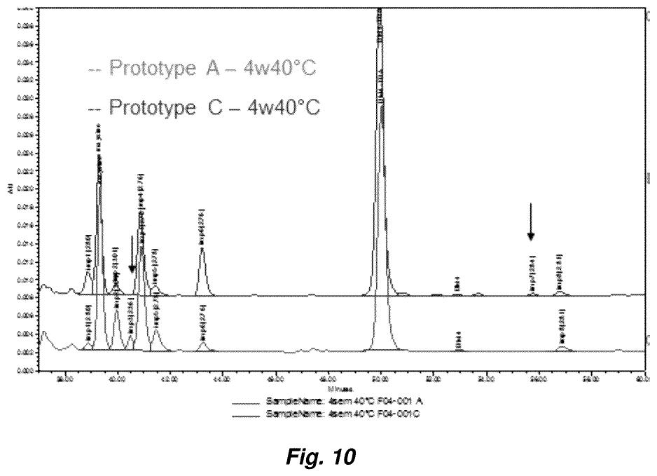

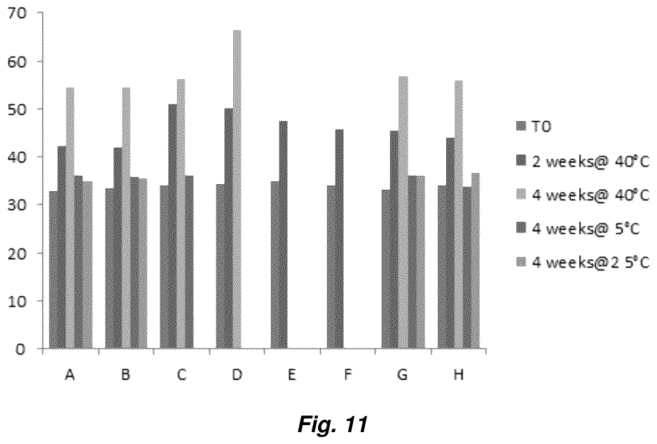

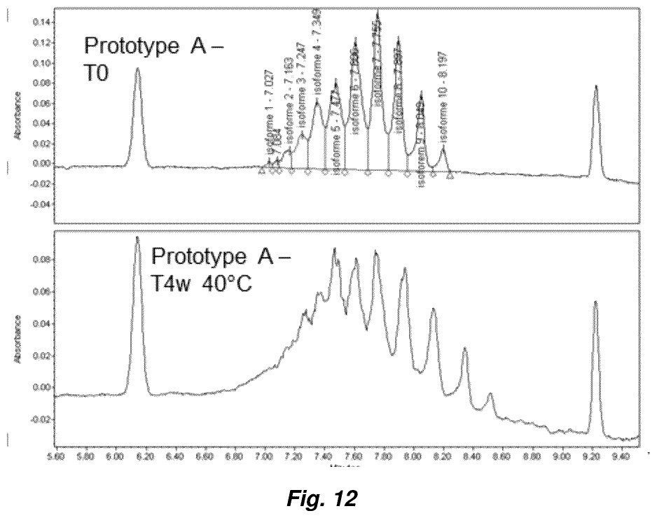

Some differences between prototypes could be observed after 2W and 4W 40° C.:
After 4 weeks at 40° C., the DAR decrease is the highest for formulation D (Histidine buffer at pH 6.5) and is high for formulations at pH 6.5 or in histidine (FIG. 6).
After 2 weeks at 40° C., the highest HMW increase is observed for citrate pH 6.0 (prototype E) and is high for the 2 other formulations above pH 5.5 (D & F). Regarding prototypes at pH 5.5 in acetate buffer (A, B, G, H), an increase of aggregates (FIG. 7) and of fragments (FIG. 8) was observed after 4W40° C., higher for formulations G and H. (Prototypes C, D, E, F not analyzed at 4W40° C.).
After 2 weeks at 40° C., regarding the free maytansinoids analysis, formulations D, E and F presented the higher levels of free maytansinoids. After 4 weeks at 40° C., only formulations at pH 5.5 were analyzed (prototypes A, B, C, G, and H) and prototype A presented the lower level of free maytansinoids (FIG. 9). The buffer type (acetate versus histidine) influenced maytansinoid detected impurities (FIG. 10).
After 4 weeks at 40° C., the highest increase of OD at 350 nm is observed for prototype D (histidine, pH 6.5) and is high for formulations at high pH or in histidine.
Concerning results of charge isoforms by iCIEF, the highest increase of acidic species is observed for formulation D (pH 6.5) after 4W40° C. (FIG. 12). Other tested prototypes (prototypes A, B, G, and H) behaved similarly after 2W40° C. and 4W40° C. (FIG. 11).

### Shaking Stress

After shaking stress, prototypes were similar for pH, concentration & DAR, DLS, turbidity, SEC-UPLC, Free maytansinoids.

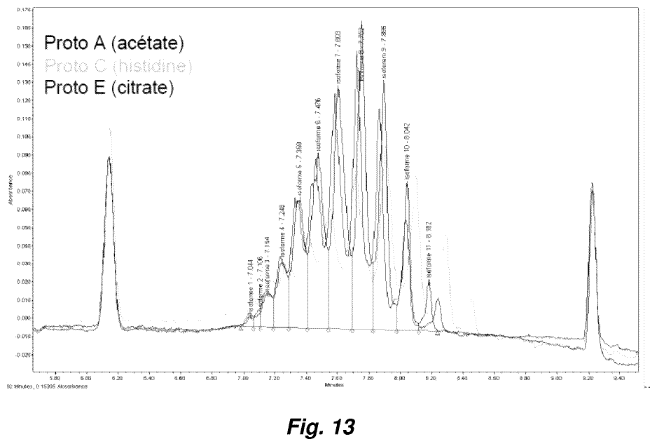

After shaking stress, some differences between prototypes were observed:
regarding visual inspection, prototypes A and B presented no particles while prototypes D, G and H contained numerous visible particles.
regarding FCM, a slight increase of particles≥10 and 25 μm was observed for prototype H, that could show an aggregation start with PS20 (Table 5).
regarding icIEF results, prototype C (histidine, pH 5.5) presents the highest charge variants evolution (+5% acids, −3% basics, PI shift, FIG. 13). Other prototypes depict no charge variants evolution after shaking stress.

TABLE 5
FCM results after shaking: Number of particles/mL

| Formu- | T0 | | | Shaking | | |
|---|---|---|---|---|---|---|
| lation | ≥2 μm | ≥10 μm | ≥25 μm | ≥2 μm | ≥10 μm | ≥25 μm |
| A | 138 | 13 | 6 | 263 | 35 | 15 |
| B | 72 | 6 | 1 | 165 | 29 | 9 |
| C | 128 | 11 | 7 | 178 | 34 | 10 |
| D | 194 | 23 | 5 | 653 | 18 | 4 |
| E | 236 | 17 | 7 | 229 | 18 | 2 |
| F | 100 | 5 | 2 | 390 | 18 | 6 |
| G | 83 | 2 | 1 | 268 | 32 | 6 |
| H | 133 | 10 | 1 | 413 | 149 | 50 |

### Freeze and Thaw (F/T) Stress

After F/T stress, prototypes were similar for visual inspection, pH, concentration & DAR, turbidity, SEC-UPLC, Free Maytansinoids, FCM and DLS. Regarding icIEF, prototype C is the most sensitive to F/T stress (+6% acids for −20° C. and +5% for −80° C., −6% basics for the 2 temperatures, Table 6, Table 7).

TABLE 6
iCIEF results after F/T −20° C./RT

| | Main | % Main | Σ % | Σ % | No. isoforms |
|---|---|---|---|---|---|
| Formulation | pI | Peak | Acids | Basics | observed |
| A | 7.62 | 20.5 | 34.1 | 45.4 | 11 |
| B | 7.61 | 20.6 | 33.2 | 46.3 | 11 |
| C | 7.62 | 20.2 | 40.1 | 39.1 | 11 |
| D | 7.60 | 20.2 | 35.8 | 44.1 | 11 |
| E | 7.56 | 20.3 | 34.6 | 45.1 | 11 |
| F | 7.58 | 20.6 | 34.5 | 44.9 | 11 |
| G | 7.57* | 20.0 | 34.8 | 45.3 | 11 |
| H | 7.58 | 20.4 | 34.3 | 45.4 | 11 |

*2^{nd} main isoform but kept for % consistency between conditions

### Conclusion

TABLE 7
iCIEF results after F/T −80° C./RT

| | Main | % Main | Σ % | Σ % | No. isoforms |
|---|---|---|---|---|---|
| Formulation | pI | Peak | Acids | Basics | observed |
| A | 7.63 | 20.8 | 33.8 | 45.4 | 11 |
| B | 7.62 | 20.5 | 34.6 | 44.6 | 11 |
| C | 7.62 | 20.6 | 38.8 | 40.6 | 11 |
| D | 7.62 | 19.6 | 35.6 | 44.8 | 11 |
| E | 7.56 | 20.1 | 35.2 | 44.7 | 11 |
| F | 7.57 | 21.2 | 34.3 | 44.5 | 11 |
| G | 7.56 | 20.3 | 34.7 | 45.0 | 11 |
| H | 7.56* | 20.3 | 35.2 | 44.6 | 11 |

*2^{nd} main isoform but kept for % consistency between conditions

The yield after UF/DF concentrated up to around 12 mg/mL was better in Acetate buffer (˜92%) and the processing time was shorter than in citrate buffer. huMAb2-3-SPDB-DM4 in Citrate 10 mM pH 6.0 buffer was slightly opalescent post UF/DF.

### Regarding Formulations Behavior Upon Stress:

A very slight opalescence was observed by visual inspection for all formulations but didn't evolve. By OD 350 nm, the turbidity increased after 40° C. stress for all formulations but mainly for histidine pH 6.5 (formulation D).

Some visible particles were observed only after stress of shaking for all formulations but less for formulations A and B (acetate pH 5.5 with sucrose and mannitol).

Histidine pH 6.5 was the more destabilizing for DAR after 4 weeks 40° C.

Acetate pH 5.5 was the more stabilizing for HMW (by SEC-UPLC) after 2 weeks 40° C.

After 4 weeks 40° C., LMW (by SEC-UPLC) increased by 3 for all acetate pH 5.5 formulations (not analyzed for other formulations).

For free maytansinoid and iCIEF, pH 5.5 and acetate buffer were the more stabilizing after stress (mainly thermal stress).

No aggregation in subvisible particles with PS80 was observed by FCM.

The higher Tm (by DSC) was obtained in acetate buffer (pH 5.5 and 6.0).

In conclusion, Acetate pH 5.5 appeared to be the best stabilizing buffer (mainly for DAR, HMW, free maytansinoid, charge variants) and the presence of PS80 allowed the decrease of particles.

### Example 2. Selection of Sorbitol as Excipient

The formulation development study described in this example was designed based on the results of Example 1 with the following rationale:
Sodium Acetate buffer 10 mM at pH 5.5 was selected as improving several physicochemical quality attributes (mainly DAR, HMWs %, Free Maytansinoids & charge variants);
Mannitol tested in Example 1 was kept for one formulation and other polyols were tested as replacement: sucrose and sorbitol, at concentrations compatible with isotonicity; and
PS80 was selected from Example 1 as improving aggregation propensity (mainly subvisible particles by FCM after shaking stress). The concentration of PS80 was varied between 0.01% w/v and 0.04% w/v.

The composition of the 6 prototypes selected for this study is given in Table 8.

TABLE 8
Prototype formulations

| Formulation | | Targeted | | Concentration |
|---|---|---|---|---|
| ID | Buffer system | pH | Excipient | (mg/mL) |
| A | Acetate 10 mM | 5.5 | Sucrose 2.5% + | 5.0 |
| | | | Mannitol 3.75% + | |
| | | | PS80 0.01% | |
| B | Acetate 10 mM | 5.5 | Sucrose 10% + | 5.0 |
| | | | PS80 0.01% | |
| C | Acetate 10 mM | 5.5 | Sucrose 10% + | 5.0 |
| | | | PS80 0.02% | |
| D | Acetate 10 mM | 5.5 | Sucrose 10% + | 5.0 |
| | | | PS80 0.04% | |
| E | Acetate 10 mM | 5.5 | Sorbitol 5% + | 5.0 |
| | | | PS80 0.01% | |
| F | Acetate 10 mM | 5.5 | Sorbitol 5% + | 5.0 |
| | | | PS80 0.04% | |

The 6 prototype formulations were stored at 40° C. up to 1 month, at −20° C., 5° C. and 25° C. up to 3 months. They were also evaluated under stress conditions by shaking stress followed by in-use simulation. Table 9 describes the time points, stress conditions and analytical methods of the formulation study.

TABLE 9
Timepoints and analytical methods

| Timepoints | Purpose | Analytical methods |
|---|---|---|
| T0 | Initial timepoint | DSC, Visual observation, |
| | | turbidity, pH, DAR and |
| | | protein concentration, |
| | | icIEF, Free Maytansinoids, |
| | | SEC-UPLC (Monomer %, |
| | | HMWs %, LMWs %), |
| | | DLS, FCM, Binding by |
| | | ELISA, SDS-PAGE under |
| | | reduced and non-reduced |
| | | conditions |
| Shaking stress | Orbital shaking, 15 hours | Visual observation, LO |
| (SS) | 350 rpm | (HIAC) and FCM |
| In-use simulation | Samples were diluted in | Visual observation, LO |
| (performed after | NaCl 0.9% Polyolefine | (HIAC), DAR and protein |
| SS) | bags at a concentration of | concentration, SEC-UPLC |
| | 0.8 mg/mL (P0 analysis). | (Monomer %, HMWs %, |
| | Diluted bags were kept | LMWs %) |
| | 24 h at room temperature | |
| | (P1 analysis). | |
| | The diluted bags were then | |
| | perfused via Hospira | |
| | infusion set (Polyethylene | |
| | tube and polyethersulfone | |
| | 0.2 μm filter) and pumps | |
| | at 1 mL/min. The collected | |
| | samples (around 10 mL) | |
| | were analyzed (P2 | |
| | analysis). | |

Drug substance used in this Example was derived from a single batch and was characterized by formulation in Acetate 10 mM, pH 5.5, 12.4 g/L. Samples were diluted in acetate buffer and supplemented with excipients to arrive at the 6 formulations shown in Table 8.

Visual inspection for appearance (clarity and particles) was performed on a visual inspection table. Vials were inspected 5 s on white surface then on black surface and the presence of visible particles was assessed. Vials were additionally inspected using an optical fiber MLC-150 C from MOTIC (color temperature: 2000-3500K), with a scoring detailed in Table 10 and Table 11.

TABLE 10
Visual inspection scoring for opalescence evaluation

| | Opalescence |
|---|---|
| L | Limpid |
| SO | Slightly opalescent |
| O | Opalescent |

TABLE 11
Visual inspection scoring for particles evaluation

| Detection of particles | | | Number of particles |
|---|---|---|---|
| 0 | Not detected with cold light (optical fiber) | + | Countable (1 ≤ n ≤ 5) |
| 1 | Detected only with cold light (optical fiber) | ++ | Several (n > 5) |
| 2 | visible particle on visual inspection table | +++ | Numerous |

### Protein Concentration and DAR (UV)

Protein Concentration and DAR measurement was performed by UV measurement, using the following method: samples were diluted by a factor 10, then the OD at 280 nm and 254 nm were measured. Below formula are used to calculate the DAR and protein concentration.

$$[ Drug ] ( M ) = A 254 - ( 0.43 \times A 280 ) / ( 4.83 \times 4927 ) - ( 0.43 \times 4927 )$$

$$[ Drug ] mg / mL = [ Drug ] ( M ) \times 780 \times F$$

$$[ Protein ] ( M ) = A 280 - 4927 \times [ Drug ] ( M ) / 201400$$

$$[ Protein ] mg / mL = [ Protein ] ( M ) \times 144522 \times F$$

$$DAR = [ Drug ] ( M ) / [ Protein ] ( M )$$

### Turbidity

Turbidity was assessed by measuring the absorbance of the solution at 350 nm on 0.8 mL of non-diluted solution on a Cary UV 100 spectrometer.

### Light Obscuration

Sub-visible particulate counting was performed by light obscuration technique using a high accuracy liquid particle counter (HIAC). Each result was the average of 4 measurements of 0.9 mL of sample. About 4 mL of sample was needed to perform the analysis.

### Protein Purity by SDS-PAGE

Protein purity by SDS-PAGE was performed according to below procedure. Sample preparation is described in Table 12.

TABLE 12
Sample preparation for SDS PAGE

| | Reducing | Non-Reducing |
|---|---|---|
| | Conditions | Conditions |
| Sample (solution or | 5 μL | 5 μL |
| reference at 5 mg/mL) | | |
| Nu page LDS Buffer (×4) | 12.5 μL | +nem 12.5 μL |
| Reducing agent (×10) | 5 μL | — |
| Formulation buffer | 20 μL | 20 μL |
| Water | 7.5 μL | 12.5 μL |
| Heating | 10 minutes at 90° C. | 10 minutes at 70° C. |
| Deposit | 20 μL | 10 μL |

The sample was diluted to a concentration of 1 g/L. A reference was passed along with the samples. The 1× migration Buffer was manufactured by diluting 50 mL of buffer Nu-Page MOPS SDS Buffer 20× (Invitrogen NP0001) in sufficient quantity for 1 L purified water. The gel was a 4-12% Bis-Tris. Electrophoretic migration parameters were 135 volts for 90 minutes. Gels were then colored with a “Blue Rotiphorese” solution. Data processing was performed with Quantity one.

### Results

Thermal stress study (1 month 5° C., 25° C., 40° C. and 3 months 5° C., 25° C., −20° C.).

### DSC

Results of DSC at T0 are presented in Table 13.

TABLE 13
DSC results at T0

| Formulation | Td (° C.) |
|---|---|
| A—Suc 2.5% Man 3.75% | 86.4 |
| PS80 0.01% | |
| B—Suc 10% PS80 0.01% | 86.7 |
| C—Suc 10% PS80 0.02% | 86.7 |
| D—Suc 10% PS80 0.04% | 86.7 |
| E—Sorb 5% PS80 0.01% | 86.4 |
| F—Sorb 5% PS80 0.04% | 86.4 |

The thermal stability was equivalent among the 6 formulations at T0.

### Visual Inspection

Results of visual inspection are provided in Table 14 (particles) and Table 15 (clarity).

TABLE 14
Visual observation results (particles)

| Visual observation | | 5° C. | | 25° C. | | 40° C. |
|---|---|---|---|---|---|---|
| Formulation | T0 | T1M | T3M | T1M | T3M | T1M |
| A-Suc 2.5% Man | 2+ | 0 | 0 | 0 | 2+ | 0 |
| 3.75% PS80 0.01% | | | | | | |
| B-Suc 10% PS80 | 2+ | 0 | 0 | 1+ | 2+ | 0 |
| 0.01% | | | | | | |
| C-Suc 10% PS80 | 2+ | 0 | 0 | 0 | 0 | 0 |
| 0.02% | | | | | | |
| D-Suc 10% PS80 | 0 | 0 | 0 | 0 | 0 | 0 |
| 0.04% | | | | | | |
| E-Sorb 5% PS80 | 2+ | 0 | 0 | 1+ | 0 | 0 |
| 0.01% | | | | | | |
| F-Sorb 5% PS80 | 2+ | 0 | 0 | 0 | 0 | 0 |
| 0.04% | | | | | | |

TABLE 15
Visual observation results (clarity)

| Visual observation | | 5° C. | | 25° C. | | 40° C. |
|---|---|---|---|---|---|---|
| Formulation | T0 | T1M | T3M | T1M | T3M | T1M |
| A-Suc 2.5% Man | L | SO | L | SO | L | SO |
| 3.75% PS80 0.01% | | | | | | |
| B-Suc 10% PS80 | L | SO | L | SO | L | SO |
| 0.01% | | | | | | |
| C-Suc 10% PS80 | L | SO | L | SO | L | SO |
| 0.02% | | | | | | |
| D-Suc 10% PS80 | L | SO | L | SO | L | SO |
| 0.04% | | | | | | |
| E-Sorb 5% PS80 | L | SO | L | SO | L | SO |
| 0.01% | | | | | | |
| F-Sorb 5% PS80 | L* | SO | L | SO | L | SO |
| 0.04% | | | | | | |

Overall, very few particles were observed in all formulations and for all timepoints. All formulations were limpid or slightly opalescent, without significant evolution other time.

### Turbidity (OD at 350 nm)

Table 16 presents the turbidity (GD at 350 nm) results after thermal stress.

TABLE 16
Turbidity

| Turbidity | | 5° C. | | 25° C. | | 40° C. | −20° C. |
|---|---|---|---|---|---|---|---|
| Formulation | T0 | T1M | T3M | T1M | T3M | T1M | T3M |
| A-Suc 2.5% Man | 0.0145 | 0.0153 | 0.0157 | 0.0185 | 0.0294 | 0.0631 | 0.0150 |
| 3.75% PS80 0.01% | | | | | | | |
| B-Suc 10% PS80 | 0.0169 | 0.0177 | 0.0159 | 0.0260 | 0.0318 | 0.0698 | 0.0149 |
| 0.01% | | | | | | | |
| C-Suc 10% PS80 | 0.0152 | 0.0163 | 0.0155 | 0.0214 | 0.0337 | 0.0692 | 0.0157 |
| 0.02% | | | | | | | |
| D-Suc 10% PS80 | 0.0164 | 0.0167 | 0.0167 | 0.0221 | 0.0332 | 0.0681 | 0.0163 |
| 0.04% | | | | | | | |
| E-Sorb 5% PS80 | 0.0140 | 0.0152 | 0.0138 | 0.0193 | 0.0277 | 0.0618 | 0.0138 |
| 0.01% | | | | | | | |
| F-Sorb 5% PS80 | 0.0146 | 0.0152 | 0.0143 | 0.0199 | 0.0289 | 0.0599 | 0.0157 |
| 0.04% | | | | | | | |

A significant evolution was observed after T1M4° C. for all formulations. A slight difference between E and F sorbitol-based formulations and others was noted at T1M4° C. and T3M25° C., with slightly less diffusion with sorbitol-based prototypes.

pH

The results of pH are reported in Table 17.

TABLE 17
pH results

| pH | | 5° C. | | 25° C. | | 40° C. | −20° C. |
|---|---|---|---|---|---|---|---|
| Formulation | T0 | T1M | T3M | T1M | T3M | T1M | T3M |
| A-Suc 2.5% Man | 5.49 | 5.49 | 5.49 | 5.48 | 5.46 | 5.48 | 5.50 |
| 3.75% PS80 0.01% | | | | | | | |
| B-Suc 10% PS80 | 5.50 | 5.48 | 5.49 | 5.49 | 5.49 | 5.48 | 5.49 |
| 0.01% | | | | | | | |
| C-Suc 10% PS80 | 5.50 | 5.49 | 5.48 | 5.48 | 5.48 | 5.48 | 5.50 |
| 0.02% | | | | | | | |
| D-Suc 10% PS80 | 5.51 | 5.48 | 5.50 | 5.49 | 5.48 | 5.49 | 5.49 |
| 0.04% | | | | | | | |
| E-Sorb 5% PS80 | 5.48 | 5.49 | 5.49 | 5.49 | 5.50 | 5.49 | 5.50 |
| 0.01% | | | | | | | |
| F-Sorb 5% PS80 | 5.48 | 5.49 | 5.50 | 5.49 | 5.52 | 5.49 | 5.50 |
| 0.04% | | | | | | | |

No evolution of pH was observed during the stresses for all formulations.

### DAR and Concentration

The results of protein concentration and DAR are reported in Table 18.

TABLE 18
Concentration and DAR results

| | | 5° C. | | 25° C. | | 40° C. | −20° C. |
|---|---|---|---|---|---|---|---|
| Formulation | T0 | T1M | T3M | T1M | T3M | T1M | T3M |
| Concentration | | | | | | | |
| A-Suc 2.5% Man | 5.2 | 5.2 | 5.1 | 5.1 | 5.2 | 5.3 | 5.2 |
| 3.75% PS80 0.01% | | | | | | | |
| B-Suc 10% PS80 | 5.1 | 5.1 | 5.1 | 5.1 | 5.2 | 5.2 | 5.1 |
| 0.01% | | | | | | | |
| C-Suc 10% PS80 | 5.1 | 5.1 | 5.2 | 5.1 | 5.2 | 5.3 | 5.2 |
| 0.02% | | | | | | | |
| D-Suc 10% PS80 | 5.1 | 5.1 | 5.2 | 5.1 | 5.2 | 5.3 | 5.2 |
| 0.04% | | | | | | | |
| E-Sorb 5% PS80 | 5.1 | 5.1 | 5.1 | 5.1 | 5.2 | 5.2 | 5.2 |
| 0.01% | | | | | | | |
| F-Sorb 5% PS80 | 5.2 | 5.2 | 5.2 | 5.2 | 5.2 | 5.3 | 5.2 |
| 0.04% | | | | | | | |
| DAR | | | | | | | |
| A-Suc 2.5% Man | 3.5 | 3.5 | 3.4 | 3.8 | 3.1 | 2.5 | 3.4 |
| 3.75% PS80 0.01% | | | | | | | |
| B-Suc 10% PS80 | 3.5 | 3.4 | 3.4 | 3.3 | 3.1 | 2.4 | 3.5 |
| 0.01% | | | | | | | |
| C-Suc 10% PS80 | 3.5 | 3.4 | 3.5 | 3.3 | 3.2 | 2.5 | 3.5 |
| 0.02% | | | | | | | |
| D-Suc 10% PS80 | 3.5 | 3.5 | 3.6 | 3.4 | 3.2 | 2.4 | 3.6 |
| 0.04% | | | | | | | |
| E-Sorb 5% PS80 | 3.4 | 3.4 | 3.3 | 3.3 | 3.0 | 2.5 | 3.3 |
| 0.01% | | | | | | | |
| F-Sorb 5% PS80 | 3.5 | 3.5 | 3.5 | 3.4 | 3.2 | 2.5 | 3.5 |
| 0.04% | | | | | | | |

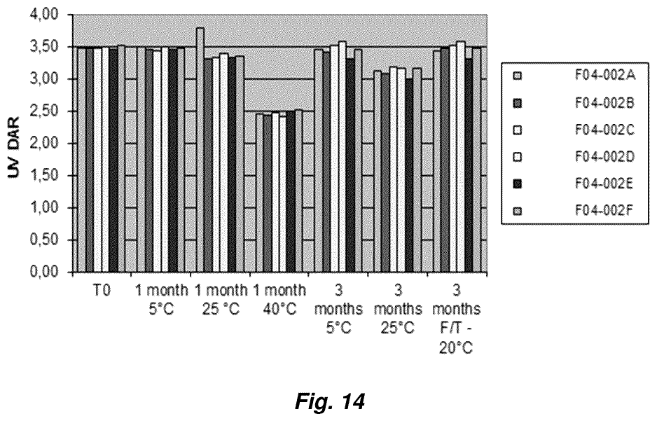

No evolution of concentration is observed during the stresses for all formulations. Regarding DAR, a decrease was observed upon thermal stress after T1M40° C. (approximately −1 unit) and T3M25° C. (approximately −0.5 unit) but no difference can be highlighted between prototypes (FIG. 14).

icIEF

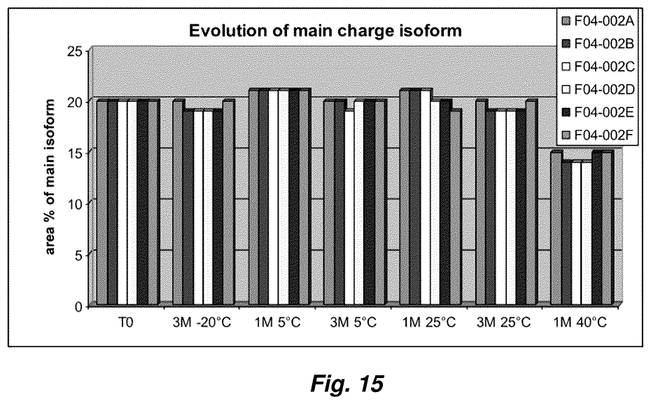

The results of Main isoform %, Acidic forms % and Basic forms % are provided in Table 19. The results of main isoform content by icIEF are depicted in FIG. 15.

TABLE 19
icIEF results

| | | | 5° C. | | 25° C. | | 40° C. | −20° C. |
|---|---|---|---|---|---|---|---|---|
| | Formulation | T0 | T1M | T3M | T1M | T3M | T1M | T3M |
| Main | A-Suc 2.5% Man 3.75% | 20 | 21 | 20 | 21 | 20 | 15 | 20 |
| Isoform % | PS80 0.01% | | | | | | | |
| | B-Suc 10% PS80 0.01% | 20 | 21 | 20 | 21 | 19 | 14 | 19 |
| | C-Suc 10% PS80 0.02% | 20 | 21 | 19 | 21 | 19 | 14 | 19 |
| | D-Suc 10% PS80 0.04% | 20 | 21 | 20 | 20 | 19 | 14 | 19 |
| | E-Sorb 5% PS80 0.01% | 20 | 21 | 20 | 20 | 19 | 15 | 19 |
| | F-Sorb 5% PS80 0.04% | 20 | 21 | 20 | 19 | 20 | 15 | 20 |
| Acidic | A-Suc 2.5% Man 3.75% | 53 | 51 | 54 | 52 | 53 | 67 | 53 |
| isoforms % | PS80 0.01% | | | | | | | |
| | B-Suc 10% PS80 0.01% | 53 | 50 | 53 | 53 | 54 | 68 | 54 |
| | C-Suc 10% PS80 0.02% | 53 | 50 | 54 | 53 | 54 | 69 | 54 |
| | D-Suc 10% PS80 0.04% | 53 | 52 | 53 | 54 | 56 | 69 | 56 |
| | E-Sorb 5% PS80 0.01% | 54 | 51 | 54 | 54 | 54 | 67 | 54 |
| | F-Sorb 5% PS80 0.04% | 54 | 52 | 54 | 53 | 54 | 67 | 54 |
| Basic | A-Suc 2.5% Man 3.75% | 27 | 28 | 27 | 27 | 27 | 18 | 27 |
| isoforms % | PS80 0.01% | | | | | | | |
| | B-Suc 10% PS80 0.01% | 27 | 29 | 27 | 27 | 27 | 18 | 27 |
| | C-Suc 10% PS80 0.02% | 27 | 29 | 27 | 26 | 27 | 18 | 27 |
| | D-Suc 10% PS80 0.04% | 27 | 28 | 28 | 26 | 24 | 17 | 24 |
| | E-Sorb 5% PS80 0.01% | 26 | 28 | 27 | 26 | 27 | 18 | 27 |
| | F-Sorb 5% PS80 0.04% | 26 | 27 | 27 | 27 | 26 | 18 | 26 |

FOR ALL FORMULATIONS AND ALL STRESS CONDITIONS EXCEPT T1M40° C., NO SIGNIFICANT DIFFERENCE IN THE MAIN ISOFORM %, ACIDIC ISOFORMS % AND BASIC ISOFORMS % WAS OBSERVED. AFTER T1M40° C., THERE WAS A SIGNIFICANT DECREASE OF MAIN AND BASIC ISOFORMS %, ALONG WITH AN INCREASE OF ACIDIC ISOFORMS %. NO SIGNIFICANT DIFFERENCE BETWEEN PROTOTYPES WAS SEEN.

FREE MAYTANSINOIDS

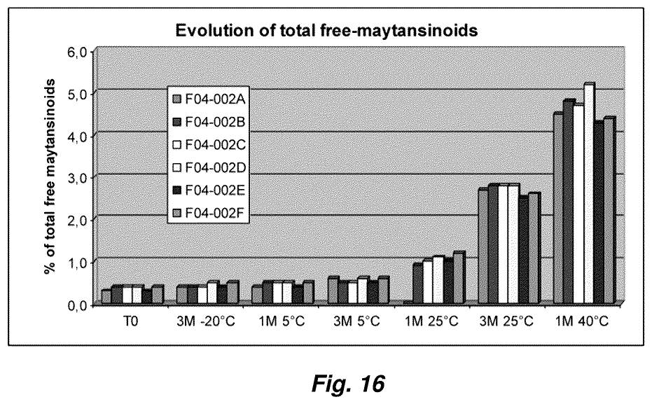

THE RESULTS OF TOTAL FREE MAYTANSINOIDS CONTENT DURING THERMAL STRESS ARE PRESENTED IN FIG. 16 AND IN TABLE 20.

TABLE 20
Free maytansinoids results

| | | | 5° C. | | 25° C. | | 40° C. | −20° C. |
|---|---|---|---|---|---|---|---|---|
| | Formulation | T0 | T1M | T3M | T1M | T3M | T1M | T3M |
| Sum of free % | A-Suc 2.5% Man 3.75% | 0.3 | 0.4 | 0.6 | 0.8 | 2.7 | 4.5 | 0.4 |
| | PS80 0.01% | | | | | | | |
| | B-Suc 10% PS80 0.01% | 0.4 | 0.5 | 0.5 | 0.9 | 2.8 | 4.8 | 0.4 |
| | C-Suc 10% PS80 0.02% | 0.4 | 0.5 | 0.5 | 1.0 | 2.8 | 4.7 | 0.4 |
| | D-Suc 10% PS80 0.04% | 0.4 | 0.5 | 0.6 | 1.1 | 2.8 | 5.2 | 0.5 |
| | E-Sorb 5% PS80 0.01% | 0.3 | 0.4 | 0.5 | 1.0 | 2.5 | 4.3 | 0.4 |
| | F-Sorb 5% PS80 0.04% | 0.4 | 0.5 | 0.6 | 1.2 | 2.6 | 4.4 | 0.5 |
| DM4-TBA (%) | A-Suc 2.5% Man 3.75% | 0.2 | 0.4 | 0.5 | 0.7 | 2.2 | 2.5 | 0.3 |
| | PS80 0.01% | | | | | | | |
| | B-Suc 10% PS80 0.01% | 0.3 | 0.4 | 0.4 | 0.8 | 2.1 | 2.5 | 0.3 |
| | C-Suc 10% PS80 0.02% | 0.3 | 0.4 | 0.4 | 0.9 | 2.2 | 2.6 | 0.3 |
| | D-Suc 10% PS80 0.04% | 0.3 | 0.4 | 0.5 | 1.0 | 2.1 | 2.9 | 0.4 |
| | E-Sorb 5% PS80 0.01% | 0.2 | 0.3 | 0.4 | 0.8 | 2.0 | 2.4 | 0.3 |
| | F-Sorb 5% PS80 0.04% | 0.3 | 0.4 | 0.5 | 1.0 | 2.1 | 2.7 | 0.4 |
| DM4-(%) | A-Suc 2.5% Man 3.75% | 0.1 | 0.1 | 0.1 | — | — | — | 0.1 |
| | PS80 0.01% | | | | | | | |
| | B-Suc 10% PS80 0.01% | 0.1 | 0.1 | 0.1 | — | — | — | 0.1 |
| | C-Suc 10% PS80 0.02% | 0.1 | 0.1 | 0.1 | — | — | — | 0.1 |
| | D-Suc 10% PS80 0.04% | 0.1 | 0.1 | 0.1 | 0.1 | — | — | 0.1 |
| | E-Sorb 5% PS80 0.01% | 0.1 | 0.1 | 0.1 | 0.1 | — | — | 0.1 |
| | F-Sorb 5% PS80 0.04% | 0.1 | 0.1 | 0.1 | 0.1 | — | — | 0.1 |

THE FREE MAYTANSINOIDS CONTENT OF ALL FORMULATIONS INCREASED AFTER T1M25° C. (SLIGHTLY), T3M25° C. AND T1M40° C. EQUIVALENT LEVELS WERE OBSERVED FOR ALL PROTOTYPES IN EACH CONDITION. FORMULATIONS E AND F PRESENTED A SLIGHTLY LOWER CONTENT OF FREE MAYTANSINOIDS AFTER T3M25° C. AND T1M4T° C. FORMULATION D HAD A SLIGHTLY HIGHER FREE MAYTANSINOIDS CONTENT AFTER T1M40° C.

OVERALL, SORBITOL-CONTAINING PROTOTYPES (FORMULATIONS E AND F) WERE BETTER REGARDING TOTAL FREE MAYTANSINOIDS CONTENT AS SEEN AT T1M40° C. AND T3M25° C.

### SEC-UPLC

Results of SEC-UPLC are depicted in Table 21.

TABLE 21
Monomer %, HMWs % and LMWs % under thermal stress by SEC-UPLC

| | | | 5° C. | | 25° C. | | 40° C. | −20° C. |
|---|---|---|---|---|---|---|---|---|
| | Formulation | T0 | T1M | T3M | T1M | T3M | T1M | T3M |
| Monomer % | A-Suc 2.5% Man 3.75% | 98.3 | 98.3 | 98.2 | 98.1 | 96.3 | 93.7 | 98.3 |
| | PS80 0.01% | | | | | | | |
| | B-Suc 10% PS80 0.01% | 98.3 | 98.3 | 98.2 | 98.1 | 96.2 | 93.7 | 98.4 |
| | C-Suc 10% PS80 0.02% | 98.3 | 98.3 | 98.2 | 98.1 | 96.2 | 93.5 | 98.4 |
| | D-Suc 10% PS80 0.04% | 98.3 | 98.3 | 98.2 | 98.1 | 96.1 | 93.5 | 98.3 |
| | E-Sorb 5% PS80 0.01% | 98.3 | 98.3 | 98.2 | 98.1 | 96.5 | 94.0 | 98.3 |
| | F-Sorb 5% PS80 0.04% | 98.3 | 98.3 | 98.2 | 98.1 | 96.4 | 93.9 | 98.3 |
| HMWs % | A-Suc 2.5% Man 3.75% | 1.7 | 1.7 | 1.7 | 1.8 | 2.1 | 3.2 | 1.7 |
| | PS80 0.01% | | | | | | | |
| | B-Suc 10% PS80 0.01% | 1.7 | 1.7 | 1.7 | 1.8 | 2.0 | 3.1 | 1.6 |
| | C-Suc 10% PS80 0.02% | 1.7 | 1.7 | 1.7 | 1.8 | 2.0 | 3.1 | 1.6 |
| | D-Suc 10% PS80 0.04% | 1.7 | 1.7 | 1.7 | 1.8 | 2.0 | 3.1 | 1.6 |
| | E-Sorb 5% PS80 0.01% | 1.7 | 1.7 | 1.7 | 1.8 | 2.0 | 3.3 | 1.7 |
| | F-Sorb 5% PS80 0.04% | 1.7 | 1.7 | 1.7 | 1.8 | 2.0 | 3.2 | 1.7 |
| LMWs % | A-Suc 2.5% Man 3.75% | 0.1 | 0.1 | 0.1 | 0.1 | 1.7 | 3.1 | <0.1 |
| | PS80 0.01% | | | | | | | |
| | B-Suc 10% PS80 0.01% | 0.1 | 0.1 | 0.1 | 0.1 | 1.8 | 3.3 | <0.1 |
| | C-Suc 10% PS80 0.02% | 0.1 | 0.1 | 0.1 | 0.1 | 1.8 | 3.4 | <0.1 |
| | D-Suc 10% PS80 0.04% | 0.1 | 0.1 | 0.1 | 0.1 | 1.8 | 3.4 | <0.1 |
| | E-Sorb 5% PS80 0.01% | 0.1 | 0.1 | 0.1 | 0.1 | 1.4 | 2.8 | <0.1 |
| | F-Sorb 5% PS80 0.04% | 0.1 | 0.1 | <0.1 | 0.1 | 1.5 | 2.8 | <0.1 |

FOR ALL FORMULATIONS AND ALL THERMAL STRESS CONDITIONS, MONOMER % AND HMWS % EVOLVED IN A SIMILAR MANNER.

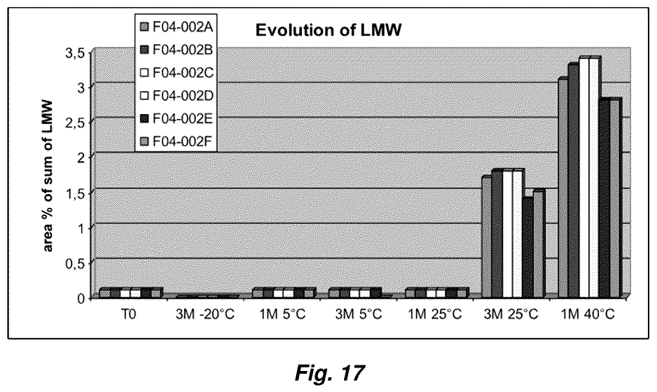

REGARDING LMWS %, SIMILAR LEVELS OF FRAGMENTS WERE OBSERVED AT T0, T1M AND T3M 5° C., T3M−20° C. AND T1M25° C. FOR ALL FORMULATIONS. AFTER T3M25° C. AND T1M40° C., FORMULATIONS E AND F SEEMED TO HAVE LOWER LMWS % THAN OTHER FORMULATIONS (−0.3% TO −0.6% AFTER T1M40° C. COMPARED TO OTHER FORMULATIONS), AS DEPICTED IN FIG. 17.

SDS PAGE

OBSERVATIONS IN NON-REDUCED CONDITIONS WERE AS FOLLOWS: AFTER T3M5° C. AND T3M25° C., SAME PATTERN AS TO AND NO DIFFERENCE BETWEEN FORMULATIONS WAS OBSERVED. AFTER T1M40° C., AN ADDITIONAL BAND APPEARED AT 46 KDA FOR ALL FORMULATIONS. ITS RELATIVE QUANTITY WAS 0.1% TO 0.6%.

OBSERVATIONS IN REDUCED CONDITIONS WERE AS FOLLOWS: AFTER T3M5° C. AND T3M25° C., SAME PATTERN AND NO SIGNIFICANT DIFFERENCE BETWEEN FORMULATIONS WAS OBSERVED. AFTER T1M40° C., UP TO 9 ADDITIONAL BANDS APPEARED AT 33, 42, 44, 83, 87, 90, 130, 150 AND 160 KDA DEPENDING ON THE FORMULATION. NO SIGNIFICANT DIFFERENCE WAS OBSERVED BETWEEN FORMULATIONS.

OVERALL, NO DIFFERENCE BETWEEN FORMULATIONS CAN BE HIGHLIGHTED BY SDS-PAGE FOR ALL TESTED THERMAL STRESS CONDITIONS.

FCM

FCM RESULTS ARE PRESENTED IN TABLE 22.

TABLE 22
FCM results during thermal stress

| Particles/ | | | 5° C. | | 25° C. | | 40° C. | −20° C. |
|---|---|---|---|---|---|---|---|---|
| mL | Formulation | T0 | T1M | T3M | T1M | T3M | T1M | T3M |
| ≥2 μm | A-Suc 2.5% Man 3.75% | 436 | 192 | 710 | 129 | 362 | 384 | 336 |
| | PS80 0.01% | | | | | | | |
| | B-Suc 10% PS80 0.01% | 446 | 126 | 642 | 192 | 525 | 321 | 131 |
| | C-Suc 10% PS80 0.02% | 211 | 371 | 491 | 144 | 495 | 1806 | 374 |
| | D-Suc 10% PS80 0.04% | 105 | 208 | 287 | 168 | 1098 | 162 | 137 |
| | E-Sorb 5% PS80 0.01% | 158 | 284 | 422 | 194 | 326 | 210 | 168 |
| | F-Sorb 5% PS80 0.04% | 161 | 180 | 511 | 698 | 346 | 508 | 108 |
| ≥10 μm | A-Suc 2.5% Man 3.75% | 40 | 15 | 15 | 10 | 10 | 20 | 13 |
| | PS80 0.01% | | | | | | | |
| | B-Suc 10% PS80 0.01% | 35 | 10 | 28 | 9 | 12 | 23 | 12 |
| | C-Suc 10% PS80 0.02% | 15 | 16 | 13 | 10 | 13 | 54 | 33 |
| | D-Suc 10% PS80 0.04% | 6 | 16 | 11 | 15 | 16 | 11 | 11 |
| | E-Sorb 5% PS80 0.01% | 7 | 21 | 11 | 13 | 14 | 10 | 13 |
| | F-Sorb 5% PS80 0.04% | 8 | 14 | 11 | 63 | 15 | 16 | 13 |
| ≥25 μm | A-Suc 2.5% Man 3.75% | 4 | 3 | 1 | 1 | 1 | 3 | 2 |
| | PS80 0.01% | | | | | | | |
| | B-Suc 10% PS80 0.01% | 1 | 1 | 5 | 1 | 2 | 4 | 1 |
| | C-Suc 10% PS80 0.02% | 2 | 1 | 2 | 2 | 2 | 5 | 2 |
| | D-Suc 10% PS80 0.04% | 1 | 1 | 1 | 4 | 1 | 1 | 1 |
| | E-Sorb 5% PS80 0.01% | 1 | 1 | 1 | 2 | 4 | 2 | 1 |
| | F-Sorb 5% PS80 0.04% | 1 | 3 | 0 | 4 | 6 | 0 | 1 |

FOR ALL PROTOTYPES AND ALL THERMAL STRESS CONDITIONS, THERE WAS NO SIGNIFICANT EVOLUTION OF THE SUB-VISIBLE PARTICLES BY FCM.

BINDING TO CEACAM5 BY ELISA

BINDING TO CEACAM5 BY ELISA RESULTS ARE PRESENTED IN TABLE 23. FORMULATIONS WERE TESTED AGAINST THE TO SAMPLES AFTER T3M −20° C., T3M5° C., AND T3M25° C. (RELATIVE POTENCIES EC₅₀ T0/EC₅₀ T3M).

TABLE 23
Binding by ELISA after T3M5° C., T3M25° C. and T3M −20° C.

| Formulation | T3M5° C. | T3M25° C. | T3M −20° C. |
|---|---|---|---|
| A—Suc 2.5% Man 3.75% | 96 | 98 | 104 |
| PS80 0.01% | | | |
| B—Suc 10% PS80 0.01% | 101 | 93 | 102 |
| C—Suc 10% PS80 0.02% | 99 | 98 | 98 |
| D—Suc 10% PS80 0.04% | 100 | 95 | 97 |
| E—Sorb 5% PS80 0.01% | 102 | 94 | 94 |
| F—Sorb 5% PS80 0.04% | 99 | 100 | 92 |

THE RELATIVE POTENCY OF THE BINDING TO CEACAM5 BY ELISA WAS STABLE AFTER T3M−20° C., T3M5° C., AND T3M25° C. UNDER THESE EXPERIMENTAL CONDITIONS, NO DIFFERENCE WAS NOTED BETWEEN FORMULATION PROTOTYPES.

SHAKING STRESS

TABLE 24 PRESENTS RESULTS OF VISUAL OBSERVATION AFTER SHAKING STRESS.

TABLE 24
Visual observation after shaking stress (particles)

| Formulation | T0 | Shaking stress |
|---|---|---|
| A—Suc 2.5% Man 3.75% PS80 0.01% | 2+ | 2+ |
| B—Suc 10% PS80 0.01% | 2+ | 2+ |
| C—Suc 10% PS80 0.02% | 2+ | 2+ |
| D—Suc 10% PS80 0.04% | 0 | 2++ |
| E—Sorb 5% PS80 0.01% | 2+ | 2+ |
| F—Sorb 5% PS80 0.04% | 2+ | 2++ |

FEW PARTICLES WERE OBSERVED FOR ALL FORMULATIONS AFTER SHAKING STRESS. PARTICLES WERE MOST OFTEN IDENTIFIED AS EXOGENOUS FIBERS.

### LO (HIAC) and FCM

TABLE 25 AND TABLE 26 PRESENT SUBVISIBLE PARTICLES RESULTS BY HIAC AND FCM AFTER SHAKING STRESS.

TABLE 25
HIAC results after shaking stress

| Particles/mL | T0 | | | After shaking stress | | |
|---|---|---|---|---|---|---|
| Formulations | ≥1.5 μm | ≥10 μm | ≥25 μm | ≥1.5 μm | ≥10 μm | ≥25 μm |
| A-Suc 2.5% Man 3.75% PS80 0.01% | 352 | 22 | 5 | 485 | 11 | 1 |
| B-Suc 10% PS80 0.01% | 114 | 10 | 4 | 222 | 13 | 2 |
| C-Suc 10% PS80 0.02% | 208 | 17 | 1 | 257 | 12 | 2 |
| D-Suc 10% PS80 0.04% | 198 | 24 | 5 | 155 | 13 | 1 |
| E-Sorb 5% PS80 0.01% | 277 | 19 | 3 | 105 | 7 | 1 |
| F-Sorb 5% PS80 0.04% | 241 | 34 | 2 | 124 | 6 | 2 |

TABLE 26
FCM results after shaking

| Particles/mL | T0 | | | After shaking stress | | |
|---|---|---|---|---|---|---|
| Prototypes | ≥2 μm | ≥10 μm | ≥25 μm | ≥2 μm | ≥10 μm | ≥25 μm |
| A—Suc 2.5% Man 3.75% PS80 0.01% | 436 | 40 | 4 | 1337 | 59 | 5 |
| B—Suc 10% PS80 0.01% | 446 | 35 | 1 | 1165 | 59 | 5 |
| C—Suc 10% PS80 0.02% | 211 | 15 | 2 | 759 | 102 | 12 |
| D—Suc 10% PS80 0.04% | 105 | 6 | 1 | 412 | 25 | 3 |
| E—Sorb 5% PS80 0.01% | 158 | 7 | 1 | 225 | 12 | 1 |
| F—Sorb 5% PS80 0.04% | 161 | 8 | 1 | 798 | 33 | 3 |

A SLIGHT INCREASE OF ≥10 μM PARTICLES WAS SEEN FOR FORMULATION C BY FCM, BUT NOT BY HIAC. OVERALL, THE STABILITY OF ALL FORMULATIONS WITH REGARDS TO SUB-VISIBLE PARTICLES AFTER SHAKING STRESS WAS SATISFACTORY.

IN-USE SIMULATION

### Visual Observation

THREE SAMPLING POINTS ARE ASSESSED:
P0: T0 after dilution at 0.8 g/L in NaCl 0.9%
P1: T24 h at RT after dilution at 0.8 g/L in NaCl 0.9%, without infusion through infusion line and filter
P2: T24 h at RT after dilution at 0.8 g/L in NaCl 0.9%, after infusion through infusion line and filter.

ALL FORMULATIONS PRESENTED VISIBLE PARTICLES AFTER DILUTION IN NACL 0.9%:

AT P0 SAMPLING POINT, FOLLOWING RANKING WAS PROPOSED: F<(BETTER THAN) D<C<B<E<A, WITH A BEING THE WORST CASE AND F SIGNIFICANTLY BETTER THAN OTHER FORMULATIONS.

At P1 sampling point, following ranking was proposed: F<D<C<E<B<A (less difference between samples that at P0)

At P2 sampling point, there are much less particles in all formulations except prototype E. Prototype E presented numerous particles; other prototypes were similar, with few particles.

TO CONCLUDE, THE DILUTION IN NACL 0.9% INCREASED THE LEVEL OF VISIBLE PARTICLES IN ALL FORMULATIONS. AFTER INFUSION (P2 SAMPLE), THE LEVEL OF PARTICLES RETURNED TO LEVELS COMPARABLE TO T0 LEVELS, EXCEPT FOR FORMULATION E WITH 100 PPM PS80.

SUB-VISIBLE PARTICLES

TABLE 27 AND TABLE 28 DEPICT THE RESULTS OF SUB-VISIBLE PARTICLES BY HIAC AFTER IN-USE SIMULATION, RESPECTIVELY IN PARTICLES/ML AND PARTICLES/CONTAINER.

TABLE 27
HIAC results after in-use simulation

| Particles/mL | T0 | | | P0 | | | P1 | | | P2 | | |
|---|---|---|---|---|---|---|---|---|---|---|---|---|
| | ≥1.5 | ≥10 | ≥25 | ≥1.5 | ≥10 | ≥25 | ≥1.5 | ≥10 | ≥25 | ≥1.5 | ≥10 | ≥25 |
| Formulations | μm | μm | μm | μm | μm | μm | μm | μm | μm | μm | μm | μm |
| A—Suc 2.5% Man 3.75% | 352 | 22 | 5 | 5836 | 245 | 5 | 4782 | 207 | 11 | 160 | 19 | 3 |
| PS80 0.01% | | | | | | | | | | | | |
| B—Suc 10% PS80 0.01% | 114 | 10 | 4 | 4072 | 86 | 2 | 1735 | 52 | 2 | 120 | 8 | 0 |
| C—Suc 10% PS80 0.02% | 208 | 17 | 1 | 2733 | 37 | 2 | 1445 | 25 | 1 | 59 | 6 | 1 |
| D—Suc 10% PS80 0.04% | 198 | 24 | 5 | 1992 | 18 | 1 | 889 | 45 | 3 | 37 | 7 | 1 |
| E—Sorb 5% PS80 0.01% | 277 | 19 | 3 | 2676 | 110 | 4 | 935 | 43 | 1 | 260 | 37 | 3 |
| F—Sorb 5% PS80 0.04% | 241 | 34 | 2 | 881 | 8 | 0 | 1430 | 32 | 2 | 62 | 4 | 1 |

TABLE 28
HIAC results after in-use simulation, expressed in particles per container
(50 mL bag)

| Particles per container | | T0 | | P0 | | P1 | | P2 | |
|---|---|---|---|---|---|---|---|---|---|
| Formulations | Specifications | ≥10 μm | ≥25 μm | ≥10 μm | ≥25 μm | ≥10 μm | ≥25 μm | ≥10 μm | ≥25 μm |
| A—Suc 2.5% Man | Before infusion, for | 1100 | 250 | 12250 | 250 | 10350 | 550 | 950 | 150 |
| 3.75% PS80 0.01% | information only. | | | | | | | | |
| B—Suc 10% PS80 | After infusion: | 500 | 200 | 4300 | 100 | 2600 | 100 | 400 | 0 |
| 0.01% | Number of | | | | | | | | |
| C—Suc 10% PS80 | particles ≥10 μm | 850 | 50 | 1850 | 100 | 1250 | 50 | 300 | 50 |
| 0.02% | ≤6000/recipient | | | | | | | | |
| D—Suc 10% PS80 | Number of particles | 1200 | 250 | 900 | 50 | 2250 | 150 | 350 | 50 |
| 0.04% | ≥25 μm | | | | | | | | |
| E—Sorb 5% PS80 | ≤600/recipient | 950 | 150 | 5500 | 200 | 2150 | 50 | 1850 | 150 |
| 0.01% | | | | | | | | | |
| F—Sorb 5% PS80 | | 1700 | 100 | 400 | 0 | 1600 | 100 | 200 | 50 |
| 0.04% | | | | | | | | | |

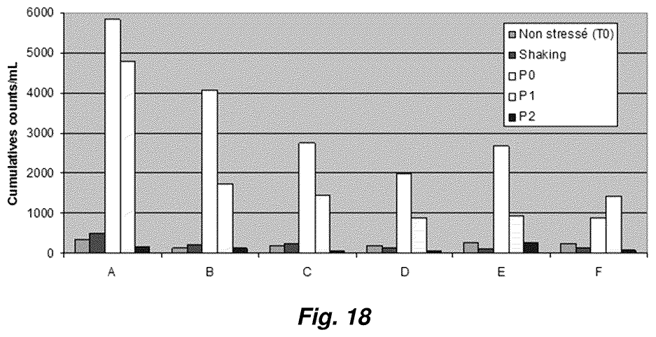

Osmotic stress by dilution in saline solution (NaCl 0.9%) generated ≥1.5 μm sub-visible particles in all formulations as seen in FIG. 18 on P0 and P1 sampling points. Formulation A had a significantly higher number of ≥1.5 μm particles. Formulations A and E had a significantly higher number of ≥10 μm particles on P0 sampling point. Both A and E formulations had the lower tested PS80 concentration of 100 ppm.

After infusion through the infusion line and 0.2 μm filter (P2 sampling point), sub-visible particles were reduced for all formulations. All formulations passed the pharmacopeia criteria for 50 mL infusion bags. Formulation E would not pass pharmacopeial criteria for volumes above 100 mL (below 25 particles/mL≥10 μm and below 3 particles/mL≥25 μm). A higher risk of non-compliance was highlighted for 100 ppm PS80 concentration.

### Concentration & DAR

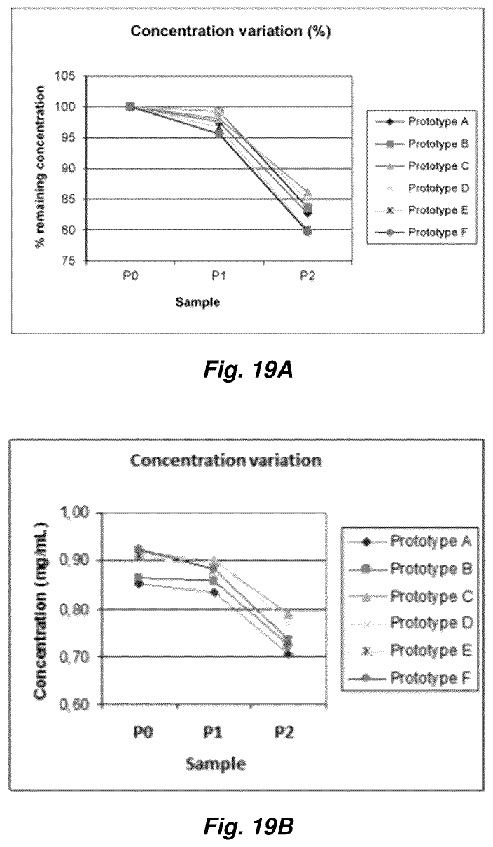

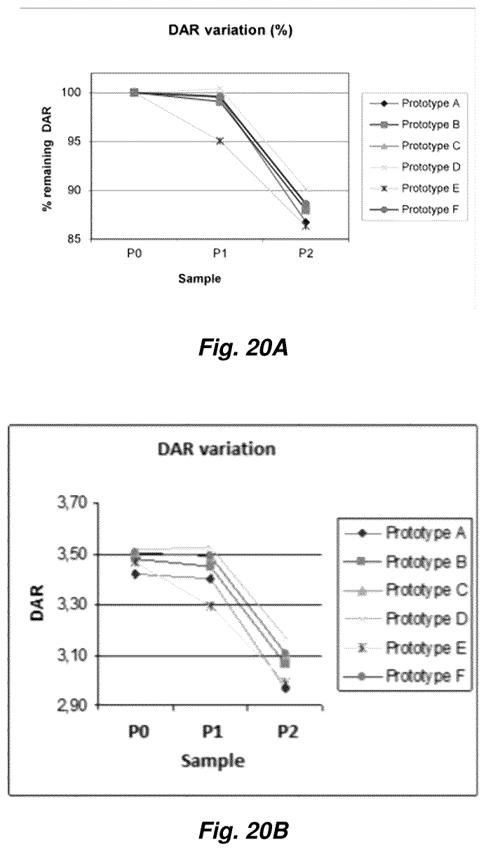

FIG. 19 AND FIG. 20 DEPICT THE RESULTS OF PROTEIN CONCENTRATION AND DAR AFTER IN-USE SIMULATION. FOR ALL FORMULATIONS, A DECREASE OF CONCENTRATION (−15 TO −20%) AND OF DAR (−10 TO −15%) WAS OBSERVED AFTER IN-USE SIMULATION OF AROUND 10 ML WITH USUAL INFUSION KIT (0.2 μM PES IN-LINE FILTER INCLUDED). NO DIFFERENCE COULD BE HIGHLIGHTED BETWEEN PROTOTYPES.

### SEC-UPLC

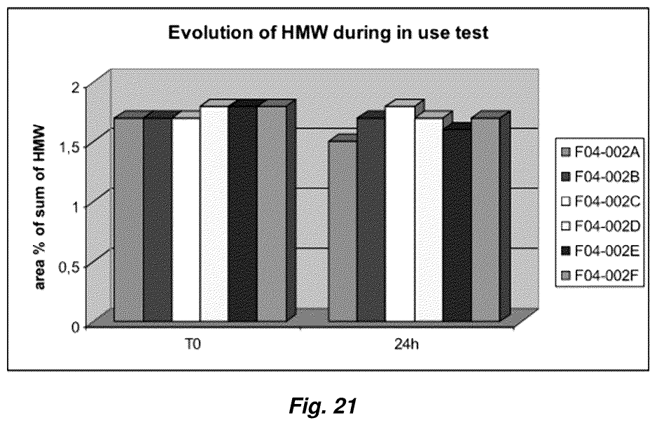

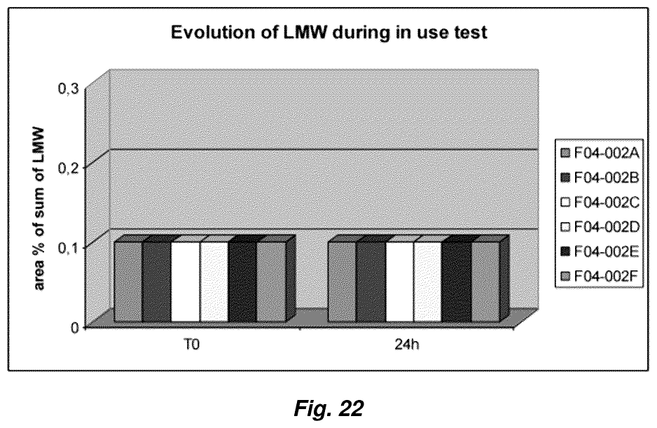

FIG. 21 and FIG. 22 present the results of HMWs % and LMWs % after in-use simulation. During in-use simulation, no significant evolution was observed by SEC for all prototypes after dilution and storage 24 h at room temperature in bag.

### Conclusion

Based on thermal stresses, sorbitol-based formulations (E & F) were better regarding Free Maytansinoids (T1M40° C.), fragmentation (T3M25° C. and T1M40° C.), and turbidity (T1M40° C.). All formulations were equivalent for other tested parameters.

ADDITIONALLY, SHAKING STRESS FOLLOWED BY IN-USE SIMULATION DEMONSTRATED THAT:

ALL FORMULATIONS SHOWED SATISFACTORY RESULTS UPON SHAKING STRESS AND NO SIGNIFICANT DIFFERENCE BETWEEN PROTOTYPES WAS HIGHLIGHTED.

Dilution in NaCl 0.9% at 0.8 mg/mL caused sub-visible particle formation, especially for formulations containing 100 ppm of PS80. Formulation E (Sorbitol 5%, PS80 100 ppm) did not pass the most strict pharmacopeia criteria by LO (HIAC).

Infusion of the diluted huMAb2-3-SPDB-DM4 at 0.8 mg/mL through the PE line+PES filter resulted in a significant decrease of protein concentration and DAR in the first milliliters.

Based on these results, an initial huMAb2-3-SPDB-DM4 formulation was selected: Na acetate 10 mM pH 5.5, sorbitol 5% (w/v), PS80 400 ppm (0.04%).

### Example 3. Impact of Addition of EDTA to Formulation of huMAb2-3-SPDB-DM4

In presence of metal traces, accelerated degradation of polysorbate may be observed. Iron, along with other metals such as copper, is known to be involved in polysorbate 80 (PS80) degradation, even when present at very low concentration (ppb). Kranz et al., J Pharm Sci 2019 108(6):2022-2032. The addition of the chelating agent disodium EDTA was investigated to evaluate its ability to limit PS80 oxidation.

### Study Design

The aim of this study was to assess the protective role of EDTA at three different concentrations (1, 10, and 50 μM) on huMAb2-3-SPDB-DM4 material that contains metal residues (representative of the reachable levels). A filling platform was used to mimic at laboratory scale the drug product (DP) filling step.

Samples were evaluated in terms of stability at 5° C., 25° C., and 40° C., up to 1 month. After formulation lock, additional analyses were performed up to 6 months on the selected EDTA concentration and comparative samples without EDTA.

TABLE 29
Test Formulations

| | | | | | Target |
|---|---|---|---|---|---|
| | | Target | | EDTA | concentration |
| Name | Buffer | pH | Excipients | content | (g/L) |
| w/o EDTA | Acetate | 5.5 | Sorbitol 5% | 0 | 5.0 |
| | 10 mM | | PS80 0.04% | | |
| EDTA 1 μM | Acetate | 5.5 | Sorbitol 5% | 1 μM | 5.0 |
| | 10 mM | | PS80 0.04% | | |
| EDTA 10 μM | Acetate | 5.5 | Sorbitol 5% | 10 μM | 5.0 |
| | 10 mM | | PS80 0.04% | | |
| EDTA 50 μM | Acetate | 5.5 | Sorbitol 5% | 50 μM | 5.0 |
| | 10 mM | | PS80 0.04% | | |

The four test formulations were stored at 5° C., 25° C., and 40° C. up to 1 month before formulation lock. Complementary analysis was performed up to 6 months at 25° C. (T6M25° C.) on a restricted number of prototypes and of analytical methods. Table 30 describes the time points, stress conditions, and analytical methods of the formulation study.

TABLE 30
Timepoints and analytical methods

| | | |
|---|---|---|
| Timepoints | Purpose | Analytical Methods |

Formulation Lock

| | | |
|---|---|---|
| T0 | Initial timepoint | Visual observation, pH, |
| | | osmolality, protein concentration, |
| | | DAR, SEC, DLS, MFI, EDTA |
| | | content, CGE, iCIEF, Free drugs, |
| | | PS80, PTMs |
| T1M5° C. | DP storage condition | Visual observation, pH, |
| | | osmolality, protein concentration, |
| | | DAR, SEC, DLS, MFI, CGE, |
| | | iCIEF, Free drugs, PS80 |
| T1M25° C. | DP accelerated condition | Visual observation, pH, |
| | | osmolality, protein concentration, |
| | | DAR, SEC, DLS, MFI, CGE, |
| | | iCIEF, Free drugs, PS80 |
| T2W40° C. | DP stress condition | Visual observation, pH, |
| | | osmolality, protein concentration, |
| | | DAR, SEC, DLS, MFI, CGE, |
| | | iCIEF, Free drugs, PS80, PTMs |
| T1M40° C. | DP stress condition | Visual observation, pH, |
| | | osmolality, protein concentration, |
| | | DAR, SEC, DLS, MFI, CGE, |
| | | iCIEF, Free drugs, PS80, PTMs |

Complementary Analysis

| | | |
|---|---|---|
| T3M5° C. | Focus on selected | Visual observation, pH, |
| | formulation and without | osmolality, protein concentration, |
| | EDTA comparative | DAR, SEC, DLS, MFI, PS80 |
| | sample | |
| T3M25° C. | Focus on selected | Visual observation, pH, |
| | formulation and without | osmolality, protein concentration, |
| | EDTA comparative | DAR, SEC, DLS, MFI, PS80 |
| | sample | |
| T6M25° C. | Focus on selected | PS80 |
| | formulation and without | |
| | EDTA comparative | |
| | sample | |

The drug substance (DS) used for this study contained 24 ng/mL of Iron and 7 ng/mL of Ni. The DS as so provided was formulated in Acetate 10 mM, Sorbitol 5%, PS80 0.04%, pH 5.5, 5 g/L, and was stored at −20° C.

### Methods

### Preparation of Primary Packaging Materials

Glass vials were manually washed with water for injection (WFI) and depyrogenized in oven before filling. Stoppers were sterilized by autoclaving.

### Formulations Preparation

Three EDTA stock solutions (50 μM, 500 μM, and 2500 μM) were prepared by dissolving disodium EDTA in WFI in graduated glass flasks. The obtained solutions were filtered using 0.22 μm sterilizing syringe filters.

The ADC DS was thawed, pooled in a 10 L polycarbonate (PC) bottle, and homogenized by manual inversion of the bottle. The ADC DS was then divided into six 2 L PC bottles filled at 1.5 L (1.527 kg) with a peristaltic pump.

EDTA stock solutions were then added to the ADC DS and homogenized by manual inversion of the bottles. Table 31 gathers the calculated volumes of EDTA stock solution to be added to the ADC DS, in order to obtain the following target concentration: no EDTA, 1 μM, 10 μM and 50 μM.

TABLE 31
EDTA spiking volumes

| Targeted EDTA | | | |
|---|---|---|---|
| concentration | EDTA 50 μM | EDTA 500 μM | EDTA 2500 μM |
| 0 μM | 0 | 0 | 0 |
| 1 μM | 30.6 mL | 0 | 0 |
| 10 μM | 0 | 30.6 mL | 0 |
| 50 μM | 0 | 0 | 30.6 mL |

Once prepared, solutions were stored at 5° C. until the start of the DP filling. After EDTA spiking, the dilution factor of initial formulation was estimated at approximately 2%.

### Vial Filling

After formulation, the product was filtered in a 10 L intermediate bag and then filled into 10R vials on a dedicated filling platform mimicking the industrial drug product (DP) process.

The filled vials were then manually stoppered and crimped.

### Analytical Methods

### Visual Observation

Samples were visually inspected under a pharmacopeia chamber and an optical fiber MLC-150C from MOTIC to analyze the presence of visible particles (scoring detailed in Table 32).

TABLE 32
Ranking for visual observation

| | Detection of particles | | Number of particles |
|---|---|---|---|
| 0 | Not detected with cold light (optical fiber) | + | Countable (1 ≤ n ≤ 5) |
| 1 | Detected only with cold light (optical fiber) | ++ | Several (n > 5) |
| 2 | visible particle on visual inspection table | +++ | Numerous |
| | (Eur. Ph. Conditions) | | |

### Microflow Imaging (MFI)

For MFI analysis, 1 mL of samples was needed, without any dilution. The method used was the following: sample flows at 0.1 mL/min into the cell, recording starts after 0.2 mL of purge. Measures were performed at room temperature. Hellmanex 3% and MilliQ filtered water flushing were performed before and after each measurement.

### Dynamic Light Scattering

80 μL of solution was analyzed on Nanosizer (Zetasizer nano-S, Malvern) with following parameters: 3 measurements of 11 runs of 10 seconds, with an incidence angle of 173°. No dilution needed. Results are presented as average of the three measurements.

pH

Measurements were performed at room temperature, by dropping the pH probe in 2 mL of sample.

### Osmolality

Osmolality measurements were performed using a freezing-point osmometer. Measures were repeated 3 times and results are presented as average. A volume of 20 μL was needed for each measure.

### PS80 Content

PS80 content was measured by gas chromatography (GC).

### Results and Discussion

Results discussed below are restricted to stress conditions 40° C. and 25° C. as no difference and no evolution between samples were observed on the 5° C. storage condition. Formulation lock was performed after T1M results and it was decided to only analyze the selected formulation of 10 μM and without EDTA comparison sample for later timepoints.

### Visual Observation

Visual observation results are presented in Table 33.

According to the Eu. Ph. method, all formulations were considered essentially free from visible particles at all stress points except T2W40° C. The presence of visible particles was not confirmed at T1M40° C. and was therefore attributed to non-GMP environment preparation.

When using the optical fiber method (more sensitive than the Eu. Ph. method), some particles were observed in all formulations and at all timepoints, without trend.

No significant difference and evolution could be observed in presence or absence of EDTA.

### pH and Osmolality

pH and osmolality values are presented in Table 34.

For pH, no evolution was highlighted. Values remained constant and compliant with acceptance criteria (5.2-5.8). Osmolality values did not show any evolution over time for all samples, regardless EDTA concentration.

### Protein Concentration and DAR (UV)

Protein concentration and DAR results are presented in Table 35.

Protein concentration values did not show a significant evolution over time for all samples, regardless of the EDTA concentration.

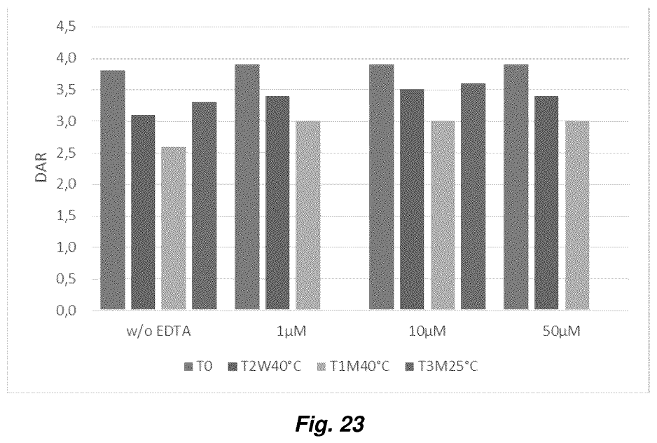

DAR values decreased over time for all samples after T2M40° C. and T1M40° C. (FIG. 23).

A higher decrease over time was observed for samples without EDTA (−1.2 at T1M40° C. w/o EDTA, versus −0.9 at T1M40° C. for the three EDTA concentrations). This trend was confirmed on the 3M25° C. timepoint, with a higher decrease on the w/o EDTA sample compared to the 10 μM EDTA sample.

TABLE 33
Visual observation

| | T0 | | | | T2W40° C. | | | | T1M40° C. | | | | T1M25° C. | | | | T3M25° C. | |
|---|---|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 | 0 | 50 |
| Vis. Obs. | 1++ | 1++ | 1++ | 1++ | 1+ | 2+++ | 2++ | 2++ | 1+ | 1+++ | 1++ | 1++ | 1++ | 1++ | 1+++ | 1+++ | 1++ | 1+ |

TABLE 34
pH and osmolality

| | T0 | | | | T2W40° C. | | | | T1M40° C. | | | | T1M25° C. | | | | T3M25° C. | |
|---|---|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 | 0 | 50 |
| pH Osm. | 5.5 | 5.5 | 5.5 | 5.5 | 5.5 | 5.5 | 5.5 | 5.5 | 5.5 | 5.5 | 5.5 | 5.5 | 5.5 | 5.5 | 5.5 | 5.5 | 5.5 | 5.5 |

TABLE 35
Protein concentration and DAR

| | T0 | | | | T2W40° C. | | | | T1M40° C. | | | | T1M25° C. | | | | T3M25° C. | |
|---|---|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 | 0 | 50 |
| Conc. | 4.4 | 4.3 | 4.7 | 4.5 | 4.5 | 4.4 | 4.8 | 4.6 | 4.5 | 4.4 | 4.8 | 4.6 | 4.5 | 4.4 | 4.7 | 4.5 | 4.6 | 4.8 |
| mg/mL | | | | | | | | | | | | | | | | | | |
| DAR | 3.8 | 3.9 | 3.9 | 3.9 | 3.1 | 3.4 | 3.5 | 3.4 | 2.6 | 3.0 | 3.0 | 3.0 | 3.8 | 3.8 | 3.9 | 3.8 | 3.3 | 3.6 |

TABLE 36
Z average and PDI %

| | T0 | | | | T2W40° C. | | | | T1M40° C. | | | | T1M25° C. | | | | T3M25° C. | |
|---|---|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 | 0 | 50 |
| Zav (nm) | 11.2 | 11.2 | 11.2 | 11.3 | 11.3 | 11.2 | 11.3 | 11.4 | 11.3 | 11.2 | 11.2 | 11.2 | 11.1 | 11.0 | 11.2 | 11.0 | 11.4 | 11.2 |
| PDI % | 21.7 | 21.3 | 20.5 | 23.9 | 26.5 | 36.4 | 23.2 | 26.1 | 24.9 | 25.5 | 20.2 | 23.5 | 27.5 | 19.4 | 18.9 | 22.0 | 30.0 | 21.6 |

TABLE 37
MFI results

| | T0 | | | | T2W40° C. | | | | T1M40° C. | | | | T1M25° C. | | | | T3M25° C. | |
|---|---|---|---|---|---|---|---|---|---|---|---|---|---|---|---|---|---|---|
| EDTA μm | 0 | 1 | 10 | 50 | 0 | 1 | 10 | 50 | 0 | 1 | 10 | 50 | 0 | 1 | 10 | 50 | 0 | 50 |
| ≥2 μm | 329 | 367 | 406 | 292 | 229 | 719 | 1104 | 310 | 376 | 237 | 906 | 171 | 902 | 714 | 846 | 751 | 1381 | 1166 |
| ≥10 μm | 0 | 8 | 0 | 10 | 5 | 30 | 32 | 4 | 0 | 0 | 8 | 9 | 0 | 6 | 53 | 21 | 39 | 15 |
| ≥25 μm | 0 | 2 | 0 | 2 | 0 | 5 | 5 | 0 | 0 | 0 | 2 | 3 | 0 | 2 | 10 | 8 | 6 | 6 |

TABLE 38
Monomer and HMWs

| | T0 | | | | T2W40° C. | | | | T1M40° C. | | | | T1M25° C. | | | | T3M25° C. | |
|---|---|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 | 0 | 50 |
| Monomer | 96.6 | 96.6 | 96.6 | 96.6 | 95.4 | 96.5 | 96.2 | 96.2 | 93.2 | 95.3 | 94.8 | 95.0 | 97.2 | 97.5 | 97.3 | 97.4 | 96.1 | 96.7 |
| % | | | | | | | | | | | | | | | | | | |
| HMWs % | 1.1 | 1.1 | 1.2 | 1.1 | 2.1 | 1.8 | 2.0 | 1.9 | 3.1 | 2.1 | 2.5 | 2.3 | 1.3 | 1.2 | 1.3 | 1.3 | 1.7 | 1.5 |

TABLE 39
Reduced cGE results

| | T0 | | | | T2W40° C. | | | | T1M40° C. | | | | T1M25° C. | | | |
|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 |
| Sum of | 98.2 | 97.8 | 98.1 | 98.3 | 97.8 | 98.0 | 97.8 | 97.9 | 95.6 | 97.5 | 98.2 | 97.3 | 98.2 | 98.1 | 98.1 | 97.9 |
| light and | | | | | | | | | | | | | | | | |
| heavy chains | | | | | | | | | | | | | | | | |
| (corrected | | | | | | | | | | | | | | | | |
| area %) | | | | | | | | | | | | | | | | |

TABLE 40
Non-reduced cGE results

| | T0 | | | | T2W40° C. | | | | T1M40° C. | | | | T1M25° C. | | | |
|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 |
| Purity | 87.1 | 87.1 | 87.3 | 87.2 | 85.2 | 86.9 | 86.9 | 86.6 | 84.8 | 87.2 | 86.7 | 87.0 | 85.0 | 86.1 | 86.2 | 86.0 |
| (H2L2, | | | | | | | | | | | | | | | | |
| corrected area | | | | | | | | | | | | | | | | |
| %) | | | | | | | | | | | | | | | | |
| Main | 9.3 | 9.3 | 9.1 | 9.3 | 10.0 | 9.6 | 9.6 | 9.7 | 8.3 | 7.7 | 8.0 | 8.0 | 10.5 | 10.4 | 10.3 | 10.4 |
| fragment | | | | | | | | | | | | | | | | |
| (RMT 0.96, | | | | | | | | | | | | | | | | |
| corrected area | | | | | | | | | | | | | | | | |
| %) | | | | | | | | | | | | | | | | |
| Sum of other | 3.5 | 3.5 | 3.6 | 3.5 | 4.8 | 3.5 | 3.5 | 3.5 | 6.9 | 5.1 | 5.2 | 5.0 | 4.5 | 3.5 | 3.6 | 3.5 |
| fragments | | | | | | | | | | | | | | | | |
| (corrected | | | | | | | | | | | | | | | | |
| area %) | | | | | | | | | | | | | | | | |

TABLE 41
PS80 content

| | T0 | | | | T2W40° C. | | | | T1M40° C. | | | | T1M25° C. | | | | T3M25° C. | | T6M25° C. | | | |
|---|---|---|---|---|---|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 | 0 | 50 | 0 | 1 | 10 | 50 |
| PS80 | 310 | 323 | 332 | 317 | 282 | 298 | 329 | 315 | 241 | 311 | 333 | 319 | 300 | 309 | 331 | 331 | 233 | 390 | 103 | 317 | 333 | 317 |
| ppm | | | | | | | | | | | | | | | | | | | | | | |

TABLE 42
Low PI and main isoform by iCIEF

| | T0 | | | | T2W40° C. | | | | T1M40° C. | | | | T1M25° C. | | | |
|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 |
| Sum of high | 9 | 9 | 9 | 9 | 7 | 8 | 8 | 8 | 4 | 6 | 5 | 7 | 9 | 9 | 9 | 9 |
| pI isoforms | | | | | | | | | | | | | | | | |
| (area %) | | | | | | | | | | | | | | | | |
| Sum of | 83 | 83 | 82 | 82 | 77 | 81 | 81 | 81 | 66 | 74 | 73 | 72 | 82 | 82 | 82 | 83 |
| main | | | | | | | | | | | | | | | | |
| isoforms | | | | | | | | | | | | | | | | |
| (area %) | | | | | | | | | | | | | | | | |
| Sum of low | 8 | 8 | 9 | 8 | 15 | 11 | 11 | 10 | 30 | 20 | 22 | 21 | 9 | 9 | 9 | 9 |
| pI isoforms | | | | | | | | | | | | | | | | |
| (area %) | | | | | | | | | | | | | | | | |

TABLE 43
Total of free maytansinoids

| | T0 | | | | T2W40° C. | | | | T1M40° C. | | | | T1M25° C. | | | |
|---|---|---|---|---|---|---|---|---|---|---|---|---|---|---|---|---|
| EDTA μM | 0 | 1 | 10 | 50 | 0 | 1 | 10 | 50 | 0 | 1 | 10 | 50 | 0 | 1 | 10 | 50 |
| Total free | 0.1 | 0.1 | 0.1 | 0.1 | 1.5 | 1.4 | 1.5 | 1.4 | 2.9 | 2.7 | 2.7 | 2.6 | 0.5 | 0.6 | 0.6 | 0.6 |
| Oxidized species | − | − | − | − | − | − | − | − | + | − | − | − | − | − | − | − |
| Impurity A | ND | ND | ND | ND | 0.25 | 0.07 | 0.10 | 0.09 | 0.69 | 0.41 | 0.43 | 0.41 | ND | ND | ND | ND |
| No. other | | | | | | | | | | | | | | | | |
| maytansinoids | 0 | 0 | 0 | 0 | 1 | 1 | 0 | 0 | 8 | 5 | 4 | 4 | 0 | 0 | 0 | 0 |
| impurities ≥ LOQ | | | | | | | | | | | | | | | | |

TABLE 44
Met255 and Met431 oxidation

| | T0 | | | | T2W40° C. | | | | T1M40° C. | | | |
|---|---|---|---|---|---|---|---|---|---|---|---|---|
| EDTA μM | 0 | 1 | 10 | 50 | 0 | 1 | 10 | 50 | 0 | 1 | 10 | 50 |
| Met255 | 5 | 4 | 5 | 4 | 8 | 5 | 6 | 5 | 23 | 10 | 12 | 11 |
| oxidation | | | | | | | | | | | | |
| (%) | | | | | | | | | | | | |
| Met431 | 2 | 2 | 1 | 1 | 3 | 2 | 2 | 2 | 10 | 4 | 4 | 4 |
| oxidation | | | | | | | | | | | | |
| (%) | | | | | | | | | | | | |

### Submicronic Particles (DLS)

In Table 36 are presented the values of PdI % and Z average over time.

No trend was observed on Zav for all timepoints and all EDTA concentrations. Regarding PDI %, a slight increase was observed for most samples at 25° C. and at 40° C. timepoints. However, no clear trend could be highlighted with regards to EDTA content.

### Subvisible Particles (MFI)

Subvisible particles results are presented in Table 37.

No difference between prototypes can be seen for all timepoints. A very slight increase of sub-visible particles was observed on the T3M25° C. timepoint; this increase was similar for w/o EDTA and 10 μM EDTA samples.

### Purity and HMWs % by Size Exclusion Chromatography (SEC)

Table 38 presents results for SEC (Monomer % and HMWs %).

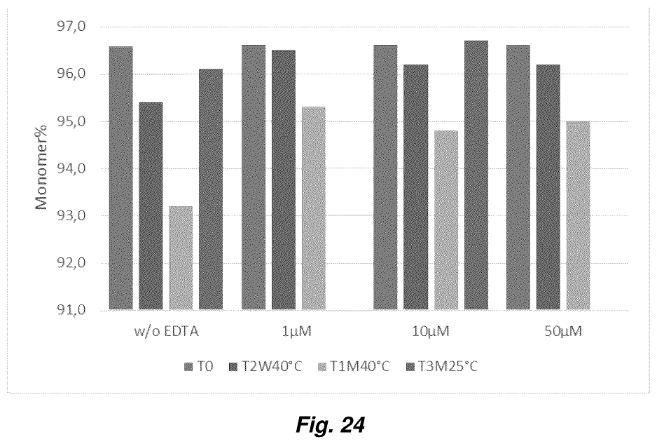

Monomer % decreased after T1M40° C. for all samples, at a faster rate in absence of EDTA (−3.4% at T1M40° C. on the w/o EDTA sample, −1.3/−1.8% on the samples with EDTA, see FIG. 24).

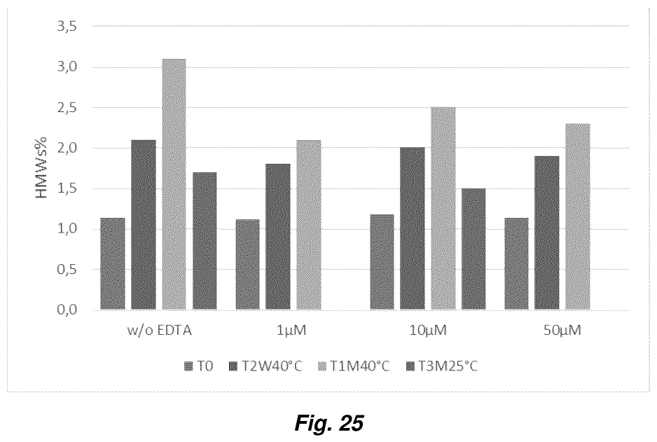

This result was confirmed by the increase of HMWs %, which is higher for the w/o EDTA sample (+2.0% at T1M40° C. on the w/o EDTA sample, +1.0/+1.3% on the with EDTA samples, see FIG. 25).

No significant differentiation can be done between the three tested EDTA concentrations on both Monomer % and HMWs %.

### Purity and Fragmentation by CGE

Table 39 and Table 40 present results of CGE in Reduced (Sum of light and heavy chains %) and Non-reduced conditions (purity %, main fragments % and Sum of other fragments %).

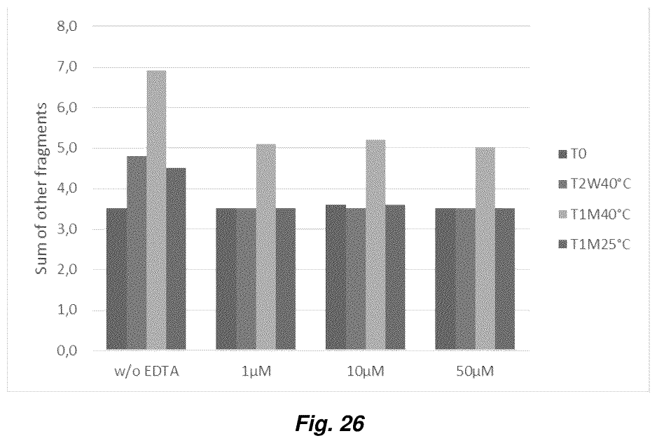

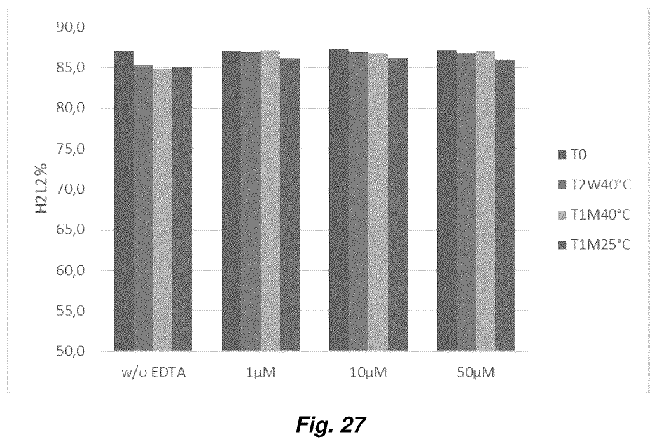

In non-reduced cGE (Table 40, FIG. 26 and FIG. 27), a significant decrease in terms of purity occurred after 1M40° C. in absence of EDTA, confirmed by an increase of fragments (sum of all fragments). With the three EDTA concentrations, no significant evolution was observed at T2W40° C., T1M25° C. and T1M40° C.

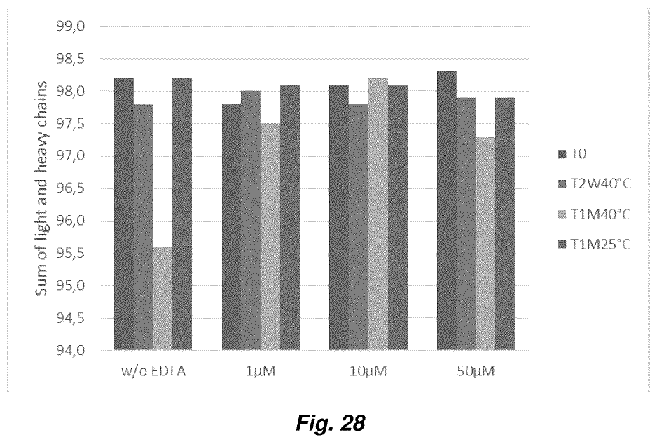

In Reduced cGE (see Table 39 and FIG. 28), a significant decrease in terms of sum of light and heavy chains occurred after 1M40° C. in absence of EDTA. With the three EDTA concentrations, no significant variation was observed at T2W40° C., T1M25° C. and T1M40° C.

### PS80 Content by Gas Chromatography (GC)

Table 41 presents results of PS80 content.

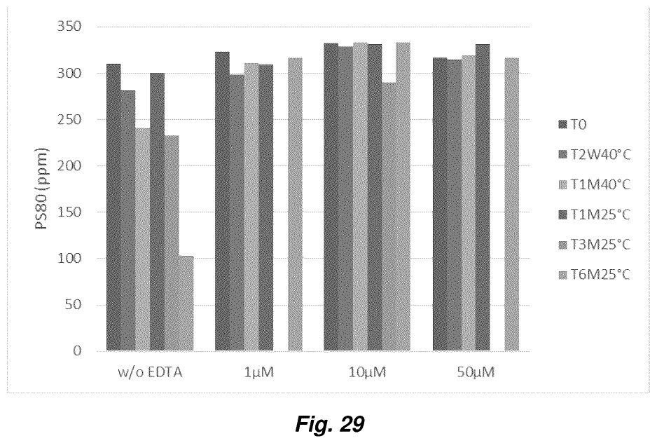

For all EDTA concentrations, no significant decrease of PS80 content was recorded up to T6M25° C. A significant decrease of PS80 was observed after T1M40° C. and T6M25° C. in absence of EDTA (see also FIG. 29).

Charge Variants Analysis by Imaged Capillary Isoelectric Focusing (iCIEF)

Table 42 gathers iCIEF results (Sum of main isoforms %, sum of high pI isoforms % and sum of low PI isoforms %).

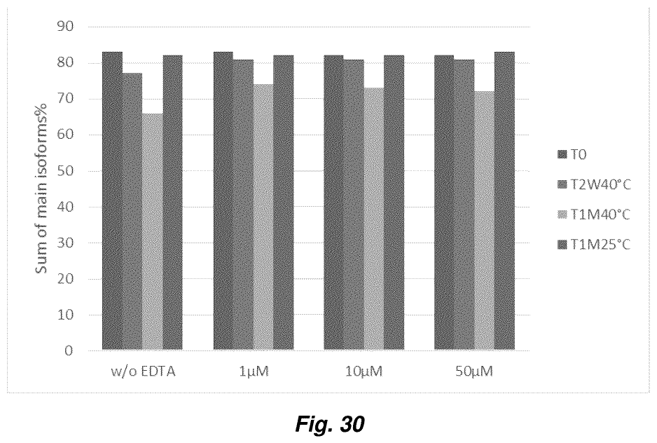

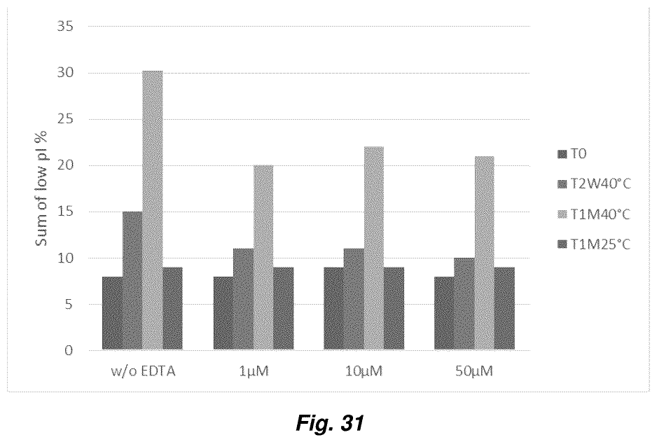

Under 40° C. stress condition, samples with EDTA had an improved stability regarding charge isoforms. The increase of sum of low pI isoforms and the decrease of main isoforms was more pronounced without EDTA (Low pI: +22% at T1M40° C. w/o EDTA, +12-13% with EDTA. Main isoforms: −17% w/o EDTA, −9/−10% with EDTA, see also FIGS. 30 and 31). No significant difference is highlighted with regards to the EDTA concentration.

### Free Maytansinoids Content

Table 43 presents the results of free drug analysis.

No significant difference can be highlighted in terms of total amount of Free drug for all samples, with and w/o EDTA. However, a difference can be seen in terms of percentage of impurity A: after 2 weeks and 1 month at 40° C., impurity A percentage was higher in absence of EDTA, in comparison with EDTA samples. The three EDTA concentrations cannot be differentiated. Also, without EDTA, oxidized species appeared after 1M40° C., while no oxidized species were detected in presence of EDTA.

### Post Translation Modifications

Only Met255 and Met431 oxidation is discussed in this section, as no difference was noticed between formulations for other PTM.

PTM analyses showed that in presence of EDTA M255 and M431 positions oxidized in a lower extent in comparison with samples w/o EDTA. No significant difference between the three EDTA concentrations could be seen (see Table 44).

### CONCLUSION

For all tested conditions, the three EDTA concentrations provided an efficient stabilization of PS80 content and of other quality attributes that may be impacted by oxidative processes (notably, PTMs, Free maytansinoids, cGE, iCIEF, and SEC).

1. A pharmaceutical formulation comprising
i) anti-human carcinoembryonic antigen-related cell adhesion molecule 5 (hCEACAM5) antibody-drug conjugate (ADC),
ii) a buffering agent,
iii) a tonicity agent, and
iv) a surfactant,
at pH about 5.0 to about 6.5,
wherein the ADC consists of huMAb2-3-SPDB-DM4.

2. The pharmaceutical formulation of claim 1, wherein the formulation is a lyophilized formulation.

3. The pharmaceutical formulation of claim 1, wherein the formulation is a liquid formulation.

4. A pharmaceutical formulation comprising
i) anti-human carcinoembryonic antigen-related cell adhesion molecule 5 (hCEACAM5) antibody-drug conjugate (ADC),
ii) a buffering agent,
iii) a tonicity agent,
iv) a surfactant, and
v) a chelating agent,
at pH about 5.0 to about 6.5,
wherein the ADC consists of huMAb2-3-SPDB-DM4.

5. The pharmaceutical formulation of claim 4, wherein the formulation is a liquid formulation.

6. The pharmaceutical formulation of claim 4, wherein the formulation is a lyophilized formulation.

7. The pharmaceutical formulation of any one of claims 1 to 6, wherein the buffering agent is selected from the group consisting of acetate, arginine, histidine, citrate, and any combination thereof.

8. The pharmaceutical formulation of claim 7, wherein the tonicity agent is a polyol.

9. The pharmaceutical formulation of claim 8, wherein the polyol is selected from the group consisting of erythritol, glycerol, lactitol, maltitol, mannitol, sorbitol, sucrose, threitol, xylitol, and any combination thereof.

10. The pharmaceutical formulation of any one of claims 1 to 9, wherein the surfactant is selected from the group consisting of polysorbate 20, polysorbate 80, poloxamer 188, and any combination thereof.

11. The pharmaceutical formulation of claim 10, wherein the surfactant is polysorbate 80.

12. The pharmaceutical composition of any one of claims 2 to 11, wherein the chelating agent is selected from the group consisting of diethyleinetriaminepentaacetic acid (DTPA), ethylene glycol-bis(β-aminoethyl ether)-N,N,N′,N′-tetraacetic acid (EGTA), ethylenediaminetetraacetic acid (EDTA), hydroxyethylenediamine triacetic acid (HEDTA), nitrilotriacetic acid, and any combination thereof.

13. The pharmaceutical composition of claim 12, wherein the chelating agent is EDTA.

14. The pharmaceutical formulation of claim 4, comprising
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof,
iv) polysorbate 80, and
v) EDTA,
at pH about 5.0 to about 6.5,
wherein the ADC consists of huMAb2-3-SPDB-DM4.

15. The pharmaceutical formulation of claim 4, comprising
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof,
iv) polysorbate 80, and
v) EDTA,
at pH about 5.0 to about 6.5,
wherein the ADC consists of huMAb2-3-SPDB-DM4.

16. The pharmaceutical formulation of claim 4, comprising
i) anti-human carcinoembryonic antigen-related cell adhesion molecule 5 (hCEACAM5) antibody-drug conjugate (ADC),
ii) a buffering agent selected from acetate, histidine, citrate, and any combination thereof,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) polysorbate 80, and
v) EDTA,
at pH about 5.0 to about 6.5,
wherein the ADC consists of huMAb2-3-SPDB-DM4.

17. The pharmaceutical formulation of claim 4, comprising
i) anti-human carcinoembryonic antigen-related cell adhesion molecule 5 (hCEACAM5) antibody-drug conjugate (ADC),
ii) a buffering agent selected from acetate, histidine, citrate, and any combination thereof, at a concentration of about 5 mM to about 100 mM,
iii) a tonicity agent selected from erythritol, glycerol, lactitol, maltitol, mannitol, sorbitol, sucrose, threitol, xylitol, and any combination thereof, at a concentration of about 1% (w/v) to about 50% (w/v),
iv) polysorbate 80, and
v) EDTA,
at pH about 5.0 to about 6.5,
wherein the ADC consists of huMAb2-3-SPDB-DM4.

18. A pharmaceutical formulation comprising
i) anti-human carcinoembryonic antigen-related cell adhesion molecule 5 (hCEACAM5) antibody-drug conjugate (ADC),
ii) sodium acetate,
iii) sorbitol,
iv) polysorbate 80, and
v) disodium EDTA,
at pH 5.5,
wherein the ADC consists of huMAb2-3-SPDB-DM4.

19. The pharmaceutical formulation of claim 18, comprising
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 1-50 μM disodium EDTA,
at pH 5.5,
wherein the ADC consists of huMAb2-3-SPDB-DM4.

20. The pharmaceutical composition of claim 19, comprising
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 1 μM disodium EDTA,
at pH 5.5,
wherein the ADC consists of huMAb2-3-SPDB-DM4.

21. The pharmaceutical composition of claim 19, comprising
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 10 μM disodium EDTA,
at pH 5.5,
wherein the ADC consists of huMAb2-3-SPDB-DM4.

22. The pharmaceutical composition of claim 19, comprising
i) 5 mg/mL anti-human carcinoembryonic antigen-related cell adhesion molecule 5 (hCEACAM5) antibody-drug conjugate (ADC),
ii) sodium acetate 10 mM,
iii) sorbitol 5% (w/v),
iv) 0.04% (w/v) polysorbate 80, and
v) 50 μM disodium EDTA,
at pH 5.5,
wherein the ADC consists of huMAb2-3-SPDB-DM4.

23. A method of treating a cancer, comprising administering to a subject in need thereof an effective amount of the pharmaceutical formulation according to any one of claims 1 to 22.

24. A method of treating a cancer, comprising administering to a subject in need thereof an effective amount of the pharmaceutical formulation according to claim 20.

25. The method according to 23 or 24, wherein the cancer is a high carcinoembryonic antigen-related cell adhesion molecule cancer.

26. The method according to any one of claims 23 to 25, wherein the cancer is selected from the group consisting of colorectal, gastric, lung, breast, prostate, ovarian, cervical, and bladder cancer.

27. The method according to any one of claims 23 to 25, wherein the cancer is selected from

[symbol]

28. The method according to any one of claims 23 to 25, wherein the cancer is lung cancer.

29. The method according to claim 28, wherein the lung cancer is non-squamous non-small cell lung carcinoma.

30. The method according to any one of claims 23 to 29, wherein the subject is a human.
